(19) United States
(12) Patent Application Publication
(10) Pub. No.: US 2016/0030426 A1
(43) Pub. Date: Feb. 4, 2016

(54) HETEROARYL COMPOUNDS AND USES THEREOF

(71) Applicant: CELGENE AVILOMICS RESEARCH, INC., Bedford, MA (US)

(72) Inventors: Richland Wayne Tester, Marlborough, MA (US); Prasoon Chaturvedi, Shrewsbury, MA (US)

(21) Appl. No.: 14/774,208

(22) PCT Filed: Mar. 14, 2013
(86) PCT No.: PCT/IB13/52048

§ 371 Date: Sep. 10, 2015

(51) Int. Cl. A61K 31/505; A61K 45/06; C12N 9/99; C07D 239/48

(52) U.S. Cl. CPC A61K 31/505; C07D 239/48; A61K 45/06; C12N 9/99

(57) ABSTRACT

The present invention provides compounds, pharmaceutically acceptable compositions thereof, and methods of using the same. Compounds of the present invention, and pharmaceutically acceptable compositions thereof, are useful for treating a variety of diseases, disorders or conditions, associated with abnormal cellular responses triggered by protein kinase-mediated events. Compounds provided by this invention are also useful for the study of kinases in biological and pathological phenoma, the study of intracellular signal transduction pathways mediated by such kinases and the comparative evaluation of new kinase inhibitors.

### FIELD OF THE INVENTION

The present invention provides compounds, and compositions thereof, useful as inhibitors of protein kinases.

### BACKGROUND OF THE INVENTION

The search for new therapeutic agents has been greatly aided in recent years by a better understanding of the structure of enzymes and other biomolecules associated with diseases. One important class of enzymes that has been the subject of extensive study is protein kinases.

Protein kinases constitute a large family of structurally related enzymes that are responsible for the control of a variety of signal transduction processes within the cell. Protein kinases are thought to have evolved from a common ancestral gene due to the conservation of their structure and catalytic function. Almost all kinases contain a similar 250-300 amino acid catalytic domain. The kinases may be categorized into families by the substrates they phosphorylate (e.g., protein-tyrosine, protein-serine/threonine, lipids, etc.).

In general, protein kinases mediate intracellular signaling by effecting a phosphoryl transfer from a nucleoside triphosphate to a protein acceptor that is involved in a signaling pathway. These phosphorylation events act as molecular on/off switches that can modulate or regulate the target protein biological function. These phosphorylation events are ultimately triggered in response to a variety of extracellular and other stimuli. Examples of such stimuli include environmental and chemical stress signals (e.g., osmotic shock, heat shock, ultraviolet radiation, bacterial endotoxin, and H₂O₂), cytokines (e.g., interleukin-1 (IL-1) and tumor necrosis factor α (TNF-α)), and growth factors (e.g., granulocyte macrophage-colony-stimulating factor (GM-CSF), and fibroblast growth factor (FGF)). An extracellular stimulus may affect one or more cellular responses related to cell growth, migration, differentiation, secretion of hormones, activation of transcription factors, muscle contraction, glucose metabolism, control of protein synthesis, and regulation of the cell cycle.

Many diseases are associated with abnormal cellular responses triggered by protein kinase-mediated events as described above. These diseases include, but are not limited to, autoimmune diseases, inflammatory diseases, bone diseases, metabolic diseases, neurological and neurodegenerative diseases, cancer, cardiovascular diseases, allergies and asthma, Alzheimer's disease, and hormone-related diseases. Accordingly, there remains a need to find protein kinase inhibitors useful as therapeutic agents.

### SUMMARY OF THE INVENTION

It has now been found that compounds of the present invention, and compositions thereof, are useful as inhibitors of one or more protein kinases and exhibit desirable characteristics for the same. Such compounds have general Formula I:

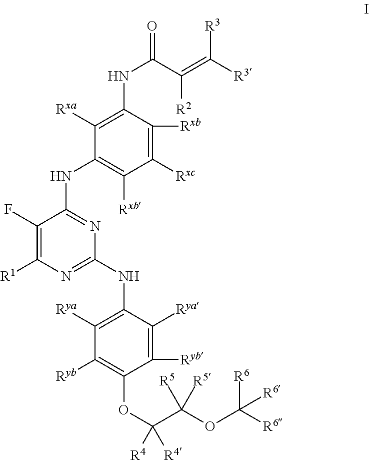

or a pharmaceutically acceptable salt thereof, wherein each of R¹, R², R³, R^{3′}, R⁴, R^{4′}, R⁵, R^{5′}, R⁶, R^{6′}, R^{6″}, R^{xa}, R^{xb}, R^{xb′}, R^{xc}, R^{ya}, R^{ya′}, R^{yb} and R^{yb′} is as defined herein.

Compounds of the present invention, and pharmaceutically acceptable compositions thereof, are useful for treating a variety of diseases, disorders or conditions, associated with abnormal cellular responses triggered by protein kinase-mediated events. Such diseases, disorders, or conditions include those described herein.

Compounds provided by this invention are also useful for the study of kinases in biological and pathological phenomena; the study of intracellular signal transduction pathways mediated by such kinases; and the comparative evaluation of new kinase inhibitors.

### DETAILED DESCRIPTION OF THE INVENTION

### 1. General Description of Certain Aspects of the Invention

As described above, the present invention provides certain compounds, and compositions thereof, useful as protein kinase inhibitors. In particular, the present invention provides certain 2,4-disubstituted pyrimidine compounds which inhibit activity of one or more protein kinases, including Bruton's tyrosine kinase (“BTK”), a member of TEC-kinases (e.g., TEC, BTK, IL2-inducible T-cell kinase (ITK), receptor-like kinases (RLK) and bone marrow kinase on chromosome X (BMX)). Such compounds have the structure of Formula I:

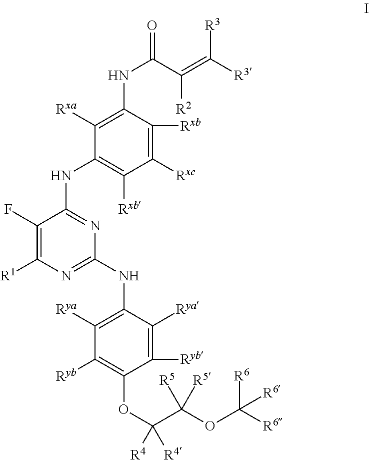

or a pharmaceutically acceptable salt thereof, wherein:
each of R¹, R², R³, R^{3′}, R⁴, R^{4′}, R⁵, R^{5′}, R⁶, R^{6′}, R^{6″}, R^{xa}, R^{xb}, R^{xb′}, R^{xc}, R^{ya}, R^{ya′}, R^{yb} and R^{yb′} is independently selected from hydrogen or deuterium;
provided that at least one of R¹, R², R³, R^{3′}, R⁴, R^{4′}, R⁵, R^{5′}, R⁶, R^{6′}, R^{6″}, R^{xa}, R^{xb}, R^{xb′}, R^{xc}, R^{ya}, R^{ya′}, R^{yb} and R^{yb′} is deuterium.

In some embodiments, the present invention provides a compound of Formula I, wherein the compound is a compound other than

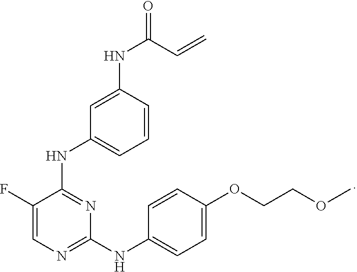 [SMILES: C=CC(=O)Nc1cccc(Nc2nc(Nc3ccc(OCCOC)cc3)ncc2F)c1]

### 2. Compounds and Definitions

Compounds of this invention include those described generally above, and are further illustrated by the classes, subclasses, and species disclosed herein. As used herein, the following definitions shall apply unless otherwise indicated. For purposes of this invention, the chemical elements are identified in accordance with the Periodic Table of the Elements, CAS version, Handbook of Chemistry and Physics, 75^{th} Ed. Additionally, general principles of organic chemistry are described in “Organic Chemistry”, Thomas Sorrell, University Science Books, Sausalito: 1999, and “March's Advanced Organic Chemistry”, 5^{th} Ed., Ed.: Smith, M. B. and March, J., John Wiley & Sons, New York: 2001, the entire contents of which are hereby incorporated by reference.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Salts derived from appropriate bases include alkali metal, alkaline earth metal, ammonium and N⁺ (C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

Unless otherwise stated, structures depicted herein are also meant to include all isomeric (e.g., enantiomeric, diastereomeric, and geometric (or conformational)) forms of the structure; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention.

As used herein, the term “inhibitor” is defined as a compound that binds to and/or inhibits the target protein kinase with measurable affinity. In certain embodiments, an inhibitor has an IC₅₀ and/or binding constant of less about 50 μM, less than about 1 μM, less than about 500 nM, less than about 100 nM, or less than about 10 nM.

The terms “measurable affinity” and “measurably inhibit,” as used herein, means a measurable change in at least one of ErbB1, ErbB2, ErbB3, ErbB4, a TEC-kinase (e.g., TEC, BTK, ITK, RLK or BMX), and/or JAK3 activity between a sample comprising a compound of the present invention, or composition thereof, and at least one of ErbB1, ErbB2, ErbB3, ErbB4, a TEC-kinase (e.g., TEC, BTK, ITK, RLK or BMX), and/or JAK3, and an equivalent sample comprising at least one of ErbB1, ErbB2, ErbB3, ErbB4, a TEC-kinase (e.g., TEC, BTK, ITK, RLK or BMX), and/or JAK3, in the absence of said compound, or composition thereof.

### 3. Description of Exemplary Compounds

Deuterium (D or ²H) is a stable, non-radioactive isotope of hydrogen and has an atomic weight of 2.0144. Hydrogen naturally occurs as a mixture of the isotopes ¹H (hydrogen or protium), D (²H or deuterium), and T (³H or tritium). One skilled in the art appreciates that the designation “hydrogen” in hydrogen-containing chemical compounds actually represents a mixture of hydrogen and about 0.015% deuterium.

Complete deuteration, or 100% deuteration, at any one site can be difficult to achieve in the laboratory. When a deuterium atom is indicated at a given site on any compound described herein, it is understood that a small percentage of hydrogen may still be present. Such compounds are said to be enriched with deuterium. Deuterium-enriched compounds are prepared via synthesis utilizing appropriately enriched starting materials. As used herein, the terms “deuterium-enriched” or “deuterium enrichment” refer to a compound, or a particular site of said compound, which comprisesdeuterium in an amount that is greater than its natural isotopic abundance (0.015%). Accordingly, in some embodiments, the present invention provides compounds comprising deuterium at a given site, wherein the percentage or level of deuterium incorporation is greater than its natural isotopic abundance.

According to one aspect, the present invention provides a compound of Formula I:

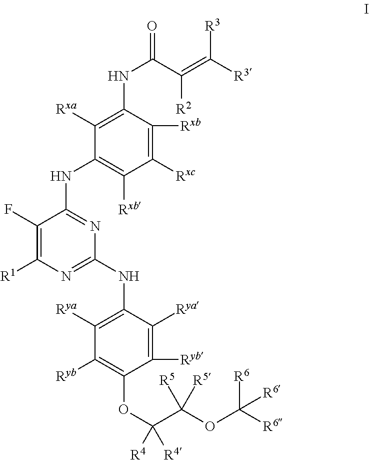

or a pharmaceutically acceptable salt thereof, wherein:
each of R¹, R², R³, R^{3′}, R⁴, R^{4′}, R⁵, R^{5′}, R⁶, R^{6′}, R^{6″}, R^{xa}, R^{xb}, R^{xb′}, R^{xc}, R^{ya}, R^{ya′}, R^{yb} and R^{yb′} is independently selected from hydrogen or deuterium;
provided that at least one of R¹, R², R³, R^{3′}, R⁴, R^{4′}, R⁵, R^{5′}, R⁶, R^{6′}, R^{6″}, R^{xa}, R^{xb}, R^{xb′}, R^{xc}, R^{ya}, R^{ya′}, R^{yb} and R^{yb′} is deuterium.

In some embodiments, R¹ is deuterium. In some embodiments, R¹ is deuterium and each of R², R³, R^{3′}, R⁴, R^{4′}, R⁵, R^{5′}, R⁶, R^{6′} and R^{6″} is hydrogen. In some embodiments, R¹ is deuterium and at least one of R², R³, R^{3′}, R⁴, R^{4′}, R⁵, R^{5′}, R⁶, R^{6′} and R^{6″} is deuterium.

In some embodiments, R¹ is hydrogen. In some embodiments, R¹ is hydrogen and at least one of R², R³, R^{3′}, R⁴, R^{4′}, R⁵, R^{5′}, R⁶, R^{6′} and R^{6″} is deuterium.

In some embodiments, R² is deuterium. In some embodiments, R² is deuterium and each of R¹, R³, R^{3′}, R⁴, R^{4′}, R⁵, R^{5′}, R⁶, R^{6′} and R^{6″} is hydrogen. In some embodiments, R² is deuterium and at least one of R¹, R³, R^{3′}, R⁴, R^{4′}, R⁵, R^{5′}, R⁶, R^{6′} and R^{6″} is deuterium.

In some embodiments, R² is hydrogen. In some embodiments, R² is hydrogen and at least one of R¹, R³, R^{3′}, R⁴, R^{4′}, R⁵, R^{5′}, R⁶, R^{6′} and R^{6″} is deuterium.

In some embodiments, R³ and R^{3′} are different. In some embodiments, R³ is hydrogen and R^{3′} is deuterium. In some embodiments, R³ is deuterium and R^{3′} is hydrogen.

In some embodiments, R³ and R^{3′} are the same. In some embodiments, each of R³ and R^{3′} is deuterium. In some embodiments, each of R³ and R^{3′} is deuterium and each of R¹, R², R⁴, R^{4′}, R⁵, R^{5′}, R⁶, R^{6′} and R^{6″} is hydrogen. In some embodiments, each of R³ and R^{3′} is deuterium and at least one of R¹, R², R⁴, R^{4′}, R⁵, R^{5′}, R⁶, R^{6′} and R^{6″} is deuterium.

In some embodiments, each of R³ and R^{3′} is hydrogen. In some embodiments, each of R³ and R^{3′} is hydrogen and at least one of R¹, R², R⁴, R^{4′}, R⁵, R^{5′}, R⁶, R^{6′} and R^{6″} is deuterium.

In some embodiments, R⁴ and R^{4′} are different. In some embodiments, R⁴ is hydrogen and R^{4′} is deuterium. In some embodiments, R⁴ is deuterium and R^{4′} is hydrogen.

In some embodiments, R⁴ and R^{4′} are the same. In some embodiments, each of R⁴ and R^{4′} is deuterium. In some embodiments, each of R⁴ and R^{4′} is deuterium and each of R¹, R², R³, R^{3′}, R⁵, R^{5′}, R⁶, R^{6′} and R^{6″} is hydrogen. In some embodiments, each of R⁴ and R^{4′} is deuterium and at least one of R¹, R², R³, R^{3′}, R⁵, R^{5′}, R⁶, R^{6′} and R^{6″} is deuterium.

In some embodiments, each of R⁴ and R^{4′} is hydrogen. In some embodiments, each of R³ and R^{3′} is hydrogen and at least one of R¹, R², R³, R^{3′}, R⁵, R^{5′}, R⁶, R^{6′} and R^{6″} is deuterium.

In some embodiments, R⁵ and R^{5′} are different. In some embodiments, R⁵ is hydrogen and R^{5′} is deuterium. In some embodiments, R⁵ is deuterium and R^{5′} is hydrogen.

In some embodiments, R⁵ and R^{5′} are the same. In some embodiments, each of R⁵ and R^{5′} is deuterium. In some embodiments, each of R⁵ and R^{5′} is deuterium and each of R¹, R², R³, R^{3′}, R⁴, R^{4′}, R⁶, R^{6′} and R^{6″} is hydrogen. In some embodiments, each of R⁵ and R^{5′} is deuterium and at least one of R¹, R², R³, R^{3′}, R⁴, R^{4′}, R^{6′} and R^{6″} is deuterium.

In some embodiments, each of R⁵ and R^{5′}, is hydrogen. In some embodiments, each of R⁵ and R^{5′} is hydrogen and at least one of R¹, R², R³, R^{3′}, R⁴, R^{4′}, R⁶, R^{6′} and R^{6″} is deuterium.

In some embodiments, R⁶, R^{6′} and R^{6″} are different. In some embodiments, each of R⁶ and R^{6′} is hydrogen and R^{6″} is deuterium. In some embodiments, each of R⁶ and R^{6″} is hydrogen and R^{6′} is deuterium. In some embodiments, each of R^{6′} and R^{6″} is hydrogen and R⁶ and is deuterium. In some embodiments, R⁶ is hydrogen and each of R^{6′} and R^{6″} is deuterium. In some embodiments, R^{6′} is hydrogen and each of R⁶ and R^{6″} is deuterium. In some embodiments, R^{6″} is hydrogen and each of R⁶ and R^{6′} is deuterium.

In some embodiments, R⁶, R^{6′} and R^{6″} are the same. In some embodiments, each of R⁶, R^{6′} and R^{6″} is deuterium. In some embodiments, each of R⁶, R^{6′} and R^{6″} is deuterium and each of R¹, R², R³, R^{3′}, R⁴, R^{4′}, R⁵ and R^{5′} is hydrogen. In some embodiments, each of R⁶, R^{6′} and R^{6″} is deuterium and at least one of R¹, R², R³, R^{3′}, R⁴, R^{4′}, R⁵ and R^{5′}, is deuterium.

In some embodiments, each of R⁶, R^{6′} and R^{6″} is hydrogen. In some embodiments, each of R⁶, R^{6′} and R^{6″} is hydrogen and at least one of R¹, R², R³, R^{3′}, R⁴, R^{4′}, R⁵ and R^{5′}, is deuterium.

In some embodiments, R^{xa} is hydrogen. In some embodiments, R^{xa} is deuterium.

In some embodiments, R^{xb} and R^{xb′} are different. In some embodiments, R^{xb} is hydrogen and R^{xb′} is deuterium. In some embodiments, R^{xb} is deuterium and R^{xb′} is hydrogen.

In some embodiments, R^{xb} and R^{xb′} are the same. In some embodiments, each of R^{xb} and R^{xb′} is hydrogen. In some embodiments, each of R^{xb} and R^{xb′} is deuterium.

In some embodiments, R^{xc} is hydrogen. In some embodiments, R^{xc} is deuterium.

In some embodiments, R^{xa}, R^{xb}, R^{xb′} and R^{xc} are the same. In some embodiments, each of R^{xa}, R^{xb}, R^{xb′} and R^{xc} is hydrogen. In some embodiments, each of R^{xa}, R^{xb}, R^{xb′} and R^{xc} is deuterium.

In some embodiments, R^{ya} and R^{ya′} are different. In some embodiments, R^{ya} is hydrogen and R^{ya′} is deuterium. In some embodiments, R^{ya} is deuterium and R^{ya′} is hydrogen.

In some embodiments, R^{ya} and R^{ya′} are the same. In some embodiments, each of R^{ya} and R^{ya′} is hydrogen. In some embodiments, each of R^{ya} and R^{ya′} is deuterium.

In some embodiments, R^{yb} and R^{yb′} are different. In some embodiments, R^{yb} is hydrogen and R^{yb′} is deuterium. In some embodiments, R^{yb} is deuterium and R^{yb′} is hydrogen.

In some embodiments, R^{yb} and R^{yb′} are the same. In some embodiments, each of R^{yb} and R^{yb′} is hydrogen. In some embodiments, each of R^{yb} and R^{yb′} is deuterium.

In some embodiments, each of R², R³ and R^{3′} is deuterium. In some embodiments, each of R², R³ and R^{3′} is hydrogen. Accordingly, in some embodiments, the present invention provides a compound of Formula II:

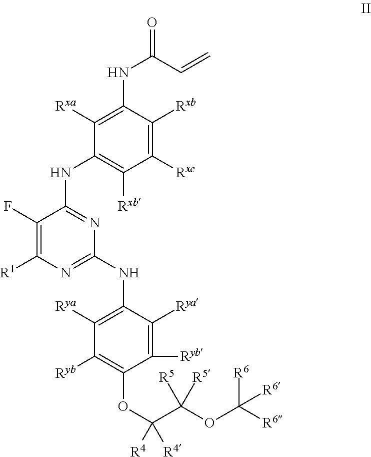

or a pharmaceutically acceptable salt thereof, wherein each of R¹, R⁴, R^{4′}, R⁵, R^{5′}, R⁶, R^{6′}, R^{6″}, R^{xa}, R^{xb}, R^{xb′}, R^{xc}, R^{ya}, R^{ya′}, R^{yb} and R^{yb′} is as defined above and described herein, provided that at least one of R¹, R⁴, R^{4′}, R⁵, R^{5′}, R⁶, R^{6′}, R^{6″}, R^{xa}, R^{xb′}, R^{xc}, R^{ya}, R^{ya′}, R^{yb} and R^{yb′} is deuterium.

In some embodiments of Formula II, at least one of R¹, R⁴, R^{4′}, R⁵, R^{5′}, R⁶, R^{6′} and R^{6″} is deuterium. In some embodiments of Formula II, the compound is selected from those in Table 1 or Table 2:

TABLE 1
Compounds of Formula II, wherein each of R¹, R^{xa}, R^{xb}, R^{xb′}, R^{xc}, R^{ya}, R^{ya′},
R^{yb} and R^{yb′} = H.

| Compound # | R⁴/R^{4′} | R⁵/R^{5′} | R⁶/R^{6′}/R^{6″} |
|---|---|---|---|
| II-1 | D | H | H |
| II-2 | H | D | H |
| II-3 | H | H | D |
| II-4 | D | D | H |
| II-5 | H | D | D |
| II-6 | D | H | D |
| II-7 | D | D | D |

TABLE 2
Compounds of Formula II, wherein R¹ = D and each of R^{xa}, R^{xb}, R^{xb′}, R^{xc},
R^{ya}, R^{ya′}, R^{yb} and R^{yb′} = H.

| Compound # | R⁴/R^{4′} | R⁵/R^{5′} | R⁶/R^{6′}/R^{6″} |
|---|---|---|---|
| II-8 | H | H | H |
| II-9 | D | H | H |
| II-10 | H | D | H |
| II-11 | H | H | D |
| II-12 | D | D | H |
| II-13 | D | H | D |
| II-14 | H | D | D |
| II-15 | D | D | D |

In some embodiments, the present invention provides a compound of Formula III:

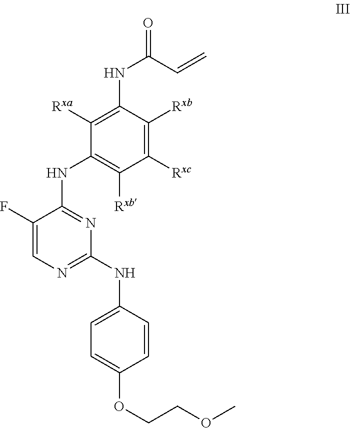

or a pharmaceutically acceptable salt thereof, wherein each of R^{xa}, R^{xb}, R^{xb′} and R^{xc} is as defined above and described herein, provided that at least one of R^{xa}, R^{xb}, R^{xb′} and R^{xc} is deuterium.

In some embodiments of Formula III, one of R^{xa}, R^{xb}, R^{xb′} and R^{xc} is deuterium. In some embodiments of Formula III, R^{xa} is deuterium. In some embodiments of Formula III, each of R^{xb} and R^{xb′} is deuterium. In some embodiments of Formula III, R^{xc} is deuterium. In some embodiments, at least two of R^{xa}, R^{xb}, R^{xb′} and R^{xc} are deuterium. In some embodiments, at least three of R^{xa}, R^{xb}, R^{xb′} and R^{xc} are deuterium.

In some embodiments of Formula III, the compound is selected from those in Table 3:

TABLE 3
Compounds of Formula III.

| Compound # | R^{xa} | R^{xb}/R^{xb′} | R^{xc} |
|---|---|---|---|
| III-1 | D | H | H |
| III-2 | H | D | H |
| III-3 | H | H | D |
| III-4 | D | D | H |
| III-5 | H | D | D |
| III-6 | D | H | D |
| III-7 | D | D | D |

In some embodiments, the present invention provides a compound of Formula IV:

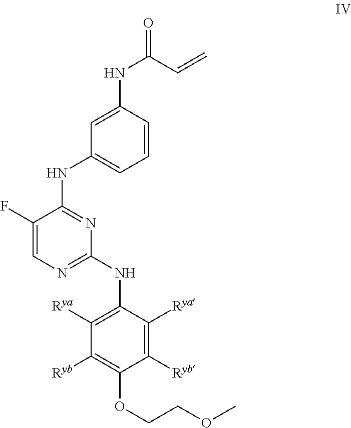

or a pharmaceutically acceptable salt thereof, wherein each of R^{ya}, R^{ya′}, R^{yb} and R^{yb′} is as defined above and described herein, provided that at least one of R^{ya}, R^{ya′}, R^{yb} and R^{yb′} is deuterium.

In some embodiments of Formula IV, one of R^{ya}, R^{ya′}, R^{yb} and R^{yb′} is deuterium. In some embodiments of Formula IV, at least two of R^{ya}, R^{ya′}, R^{yb} and R^{yb′} are deuterium. In some embodiments of Formula IV, at least three of R^{ya}, R^{ya′}, R^{yb} and R^{yb′} are deuterium. In some embodiments of Formula IV, each of R^{ya}, R^{ya′}, R^{yb} and R^{yb′} is deuterium.

In some embodiments of Formula IV, the compound is selected from those in Table 4:

TABLE 4
Compounds of Formula IV.

| Compound # | R^{ya}/R^{ya′} | R^{yb}/R^{yb′} |
|---|---|---|
| IV-1 | D | H |
| IV-2 | H | D |
| IV-3 | D | D |

In some embodiments, the present invention provides a compound of Formula V:

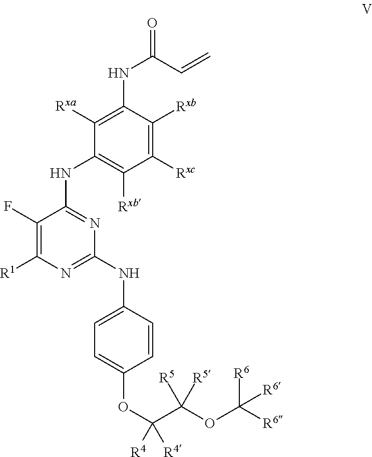

or a pharmaceutically acceptable salt thereof, wherein each of R¹, R⁴, R^{4′}, R⁵, R^{5′}, R⁶, R^{6′}, R^{6″}, R^{xa}, R^{xb}, R^{xb′} and R^{xc} is as defined above and described herein, provided that at least one of R¹, R⁴, R^{4′}, R⁵, R^{5′}, R⁶, R^{6′}, R^{6″}, R^{xa}, R^{xb}, R^{xb′} and R^{xc} is deuterium.

In some embodiments of Formula V, the compound is selected from those in Table 5, Table 6 or Table 7:

TABLE 5
Compounds of Formula V, wherein each of R⁴, R^{4′}, R⁵, R^{5′}, R⁶,
R^{6′} and R^{6″} = H.

| Compound # | R¹ | R^{xa} | R^{xb}/R^{xb′} | R^{xc} |
|---|---|---|---|---|
| V-1 | D | D | H | H |
| V-2 | D | H | D | H |
| V-3 | D | H | H | D |
| V-4 | D | D | D | H |
| V-5 | D | D | H | D |
| V-6 | D | H | D | D |
| V-7 | D | D | D | D |

TABLE 6
Compounds of Formula V, wherein R¹ = H.

| Compound # | R⁴/R^{4′} | R⁵/R^{5′} | R⁶/R^{6′}/R^{6″} | R^{xa} | R^{xb}/R^{xb′} | R^{xc} |
|---|---|---|---|---|---|---|
| V-8 | D | H | H | D | H | H |
| V-9 | D | H | H | H | D | H |
| V-10 | D | H | H | H | H | D |
| V-11 | D | H | H | D | D | H |
| V-12 | D | H | H | D | H | D |
| V-13 | D | H | H | H | D | D |
| V-14 | D | H | H | D | D | D |
| V-15 | H | D | H | D | H | H |
| V-16 | H | D | H | H | D | H |
| V-17 | H | D | H | H | H | D |
| V-18 | H | D | H | D | D | H |
| V-19 | H | D | H | D | H | D |
| V-20 | H | D | H | H | D | D |
| V-21 | H | D | H | D | D | D |
| V-22 | H | H | D | D | H | H |
| V-23 | H | H | D | H | D | H |
| V-24 | H | H | D | H | H | D |
| V-25 | H | H | D | D | D | H |
| V-26 | H | H | D | D | H | D |
| V-27 | H | H | D | H | D | D |
| V-28 | H | H | D | D | D | D |
| V-29 | D | D | H | D | H | H |
| V-30 | D | D | H | H | D | H |
| V-31 | D | D | H | H | H | D |
| V-32 | D | D | H | D | D | H |
| V-33 | D | D | H | H | D | D |
| V-34 | D | D | H | D | H | D |
| V-35 | D | D | H | D | D | D |
| V-36 | H | D | D | D | H | H |
| V-37 | H | D | D | H | D | H |
| V-38 | H | D | D | H | H | D |
| V-39 | H | D | D | D | D | H |
| V-40 | H | D | D | D | H | D |
| V-41 | H | D | D | H | D | D |
| V-42 | H | D | D | D | D | D |
| V-43 | D | H | D | D | H | H |
| V-44 | D | H | D | H | D | H |
| V-45 | D | H | D | H | H | D |
| V-46 | D | H | D | D | D | H |
| V-47 | D | H | D | D | H | D |
| V-48 | D | H | D | H | D | D |
| V-49 | D | H | D | D | D | D |
| V-50 | D | D | D | D | H | H |
| V-51 | D | D | D | H | D | H |
| V-52 | D | D | D | H | H | D |
| V-53 | D | D | D | D | D | H |
| V-54 | D | D | D | D | H | D |
| V-55 | D | D | D | H | D | D |
| V-56 | D | D | D | D | D | D |

TABLE 7
Compounds of Formula V, wherein R¹ = D.

| Compound # | R⁴/R^{4′} | R⁵/R^{5′} | R⁶/R^{6′}/R^{6″} | R^{xa} | R^{xb}/R^{xb′} | R^{xc} |
|---|---|---|---|---|---|---|
| V-57 | D | H | H | D | H | H |
| V-58 | D | H | H | H | D | H |
| V-59 | D | H | H | H | H | D |
| V-60 | D | H | H | D | D | H |
| V-61 | D | H | H | D | H | D |
| V-62 | D | H | H | H | D | D |
| V-63 | D | H | H | D | D | D |
| V-64 | H | D | H | D | H | H |
| V-65 | H | D | H | H | D | H |
| V-66 | H | D | H | H | H | D |
| V-67 | H | D | H | D | D | H |
| V-68 | H | D | H | D | H | D |
| V-69 | H | D | H | H | D | D |
| V-70 | H | D | H | D | D | D |
| V-71 | H | H | D | D | H | H |
| V-72 | H | H | D | H | D | H |
| V-73 | H | H | D | H | H | D |
| V-74 | H | H | D | D | D | H |
| V-75 | H | H | D | D | H | D |
| V-76 | H | H | D | H | D | D |
| V-77 | H | H | D | D | D | D |
| V-78 | D | D | H | D | H | H |
| V-79 | D | D | H | H | D | H |
| V-80 | D | D | H | H | H | D |
| V-81 | D | D | H | D | D | H |
| V-82 | D | D | H | H | D | D |
| V-83 | D | D | H | D | H | D |
| V-84 | D | D | H | D | D | D |
| V-85 | H | D | D | D | H | H |
| V-86 | H | D | D | H | D | H |
| V-87 | H | D | D | H | H | D |
| V-88 | H | D | D | D | D | H |
| V-89 | H | D | D | D | H | D |
| V-90 | H | D | D | H | D | D |
| V-91 | H | D | D | D | D | D |
| V-92 | D | H | D | D | H | H |
| V-93 | D | H | D | H | D | H |
| V-94 | D | H | D | H | H | D |
| V-95 | D | H | D | D | D | H |
| V-96 | D | H | D | D | H | D |
| V-97 | D | H | D | H | D | D |
| V-98 | D | H | D | D | D | D |
| V-99 | D | D | D | D | H | H |
| V-100 | D | D | D | H | D | H |
| V-101 | D | D | D | H | H | D |
| V-102 | D | D | D | D | D | H |
| V-103 | D | D | D | D | H | D |
| V-104 | D | D | D | H | D | D |
| V-105 | D | D | D | D | D | D |

In some embodiments, the present invention provides a compound of Formula VI:

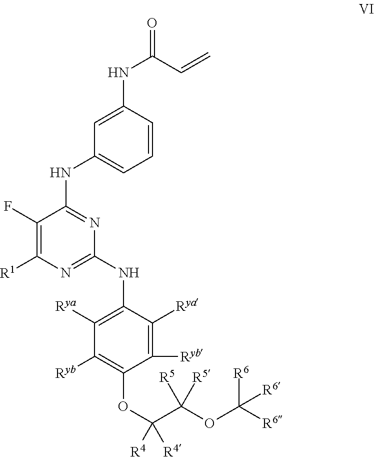

or a pharmaceutically acceptable salt thereof, wherein each of R¹, R⁴, R^{4′}, R⁵, R^{5′}, R⁶, R^{6′}, R^{6″}, R^{ya}, R^{ya′}, R^{yb} and R^{yb′} is as defined above and described herein, provided that at least one of R¹, R⁴, R^{4′}, R⁵, R^{5′}, R⁶, R^{6′}, R^{6″}, R^{ya}, R^{ya′}, R^{yb} and R^{yb′} is deuterium.

In some embodiments of Formula VI, the compound is selected from those in Table 8, Table 9 or Table 10:

TABLE 8
Compounds of Formula VI, wherein each of R⁴, R^{4′}, R⁵, R^{5′}, R⁶,
R^{6′} and R^{6″} = H.

| Compound # | R¹ | R^{ya}/R^{ya′} | R^{yb}/R^{yb′} |
|---|---|---|---|
| VI-1 | D | D | H |
| VI-2 | D | H | D |
| VI-3 | D | D | D |
| VI-4 | D | D | H |
| VI-5 | D | H | D |
| VI-6 | D | D | D |

TABLE 9
Compounds of Formula VI, wherein R¹= H.

| Compound # | R⁴/R^{4′} | R⁵/R^{5′} | R⁶/R^{6′}/R^{6″} | R^{ya}/R^{ya′} | R^{yb}/R^{yb′} |
|---|---|---|---|---|---|
| VI-7 | D | H | H | D | H |
| VI-8 | D | H | H | H | D |
| VI-9 | D | H | H | D | D |
| VI-10 | H | D | H | D | H |
| VI-11 | H | D | H | H | D |
| VI-12 | H | D | H | D | D |
| VI-13 | H | H | D | D | H |
| VI-14 | H | H | D | H | D |
| VI-15 | H | H | D | D | D |
| VI-16 | D | D | H | D | H |
| VI-17 | D | D | H | H | D |
| VI-18 | D | D | H | D | D |
| VI-19 | H | D | D | D | H |
| VI-20 | H | D | D | H | D |
| VI-21 | H | D | D | D | D |
| VI-22 | D | H | D | D | H |
| VI-23 | D | H | D | H | D |
| VI-24 | D | H | D | D | D |
| VI-25 | D | D | D | D | H |
| VI-26 | D | D | D | D | D |
| VI-27 | D | D | D | D | D |

TABLE 10
Compounds of Formula VI, wherein R¹ = D.

| Compound # | R⁴/R^{4′} | R⁵/R^{5′} | R⁶/R^{6′}/R^{6″} | R^{ya}/R^{ya′} | R^{yb}/R^{yb′} |
|---|---|---|---|---|---|
| VI-28 | D | H | H | D | H |
| VI-29 | D | H | H | H | D |
| VI-30 | D | H | H | D | D |
| VI-31 | H | D | H | D | H |
| VI-32 | H | D | H | H | D |
| VI-33 | H | D | H | D | D |
| VI-34 | H | H | D | D | H |
| VI-35 | H | H | D | H | D |
| VI-36 | H | H | D | D | D |
| VI-37 | D | D | H | D | H |
| VI-38 | D | D | H | H | D |
| VI-39 | D | D | H | D | D |
| VI-40 | H | D | D | D | H |
| VI-41 | H | D | D | H | D |
| VI-42 | H | D | D | D | D |
| VI-43 | D | H | D | D | H |
| VI-44 | D | H | D | H | D |
| VI-45 | D | H | D | D | D |
| VI-46 | D | D | D | D | H |
| VI-47 | D | D | D | H | D |
| VI-48 | D | D | D | D | D |

In some embodiments, the present invention provides a compound of Formula VII:

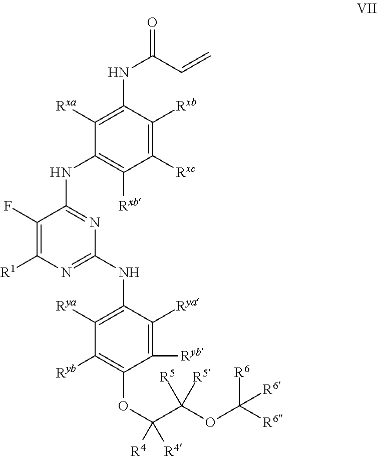

or a pharmaceutically acceptable salt thereof, wherein each of R¹, R⁴, R^{4′}, R⁵, R^{5′}, R⁶, R^{6′}, R^{6″}, R^{xa}, R^{xb}, R^{xb′}, R^{xc}, R^{ya}, R^{ya′}, R^{yb} and R^{yb′} is as defined above and described herein, provided that at least one of R¹, R⁴, R^{4′}, R⁵, R^{5′}, R⁶, R^{6′}, R^{6″}, R^{xa}, R^{xb}, R^{xb′}, R^{xc}, R^{ya}, R^{ya′}, R^{yb} and R^{yb′} is deuterium.

In some embodiments of Formula VII, the compound is selected from those in Table 11, Table 12, Table 13, Table 14, Table 15, Table 16, Table 17, Table 18 or Table 19:

TABLE 11
Compounds of Formula VII, wherein each of R¹, R⁴, R^{4′},
R⁵, R^{5′}, R⁶, R^{6′} and R^{6″} = H.

| Compound # | R^{xa} | R^{xb}/R^{xb′} | R^{xc} | R^{ya}/R^{ya′} | R^{yb}/R^{yb′} |
|---|---|---|---|---|---|
| VII-1 | D | H | H | D | H |
| VII-2 | D | H | H | H | D |
| VII-3 | D | H | H | D | D |
| VII-4 | H | D | H | D | H |
| VII-5 | H | D | H | H | D |
| VII-6 | H | D | H | D | D |
| VII-7 | H | H | D | D | H |
| VII-8 | H | H | D | H | D |
| VII-9 | H | H | D | D | D |
| VII-10 | D | D | H | D | H |
| VII-11 | D | D | H | H | D |
| VII-12 | D | D | H | D | D |
| VII-13 | D | H | D | D | H |
| VII-14 | D | H | D | H | D |
| VII-15 | D | H | D | D | D |
| VII-16 | H | D | D | D | H |
| VII-17 | H | D | D | H | D |
| VII-18 | H | D | D | D | D |
| VII-19 | D | D | D | D | H |
| VII-20 | D | D | D | H | D |
| VII-21 | D | D | D | D | D |

TABLE 12
Compounds of Formula VII, wherein each of
R⁴, R^{4′}, R⁵, R^{5′}, R⁶, R^{6′} and R^{6″} = H.

| Compound # | R¹ | R^{xa} | R^{xb}/R^{xb′} | R^{xc} | R^{ya}/R^{ya′} | R^{yb}/R^{yb′} |
|---|---|---|---|---|---|---|
| VII-22 | D | D | H | H | D | H |
| VII-23 | D | D | H | H | H | D |
| VII-24 | D | D | H | H | D | D |
| VII-25 | D | H | D | H | D | H |
| VII-26 | D | H | D | H | H | D |
| VII-27 | D | H | D | H | D | D |
| VII-28 | D | H | H | D | D | H |
| VII-29 | D | H | H | D | H | D |
| VII-30 | D | H | H | D | D | D |
| VII-31 | D | D | D | H | D | H |
| VII-32 | D | D | D | H | H | D |
| VII-33 | D | D | D | H | D | D |
| VII-34 | D | D | H | D | D | H |
| VII-35 | D | D | H | D | H | D |
| VII-36 | D | D | H | D | D | D |
| VII-37 | D | H | D | D | D | H |
| VII-38 | D | H | D | D | H | D |
| VII-39 | D | H | D | D | D | D |
| VII-40 | D | D | D | D | D | H |
| VII-41 | D | D | D | D | H | D |
| VII-42 | D | D | D | D | D | D |

TABLE 13
Compounds of Formula VII, wherein each of R⁴ and R^{4′} = D and
each of R⁵, R^{5′}, R⁶, R^{6′} and R^{6″} = H.

| Compound # | R¹ | R^{xa} | R^{xb}/R^{xb′} | R^{xc} | R^{ya}/R^{ya′} | R^{yb}/R^{yb′} |
|---|---|---|---|---|---|---|
| VII-43 | H | D | H | H | D | H |
| VII-44 | H | D | H | H | H | D |
| VII-45 | H | D | H | H | D | D |
| VII-46 | H | H | D | H | D | H |
| VII-47 | H | H | D | H | H | D |
| VII-48 | H | H | D | H | D | D |
| VII-49 | H | H | H | D | D | H |
| VII-50 | H | H | H | D | H | D |
| VII-51 | H | H | H | D | D | D |
| VII-52 | H | D | D | H | D | H |
| VII-53 | H | D | D | H | H | D |
| VII-54 | H | D | D | H | D | D |
| VII-55 | H | D | H | D | D | H |
| VII-56 | H | D | H | D | H | D |
| VII-57 | H | D | H | D | D | D |
| VII-58 | H | H | D | D | D | H |
| VII-59 | H | H | D | D | H | D |
| VII-60 | H | H | D | D | D | D |
| VII-61 | H | D | D | D | D | H |
| VII-62 | H | D | D | D | H | D |
| VII-63 | H | D | D | D | D | D |
| VII-64 | D | D | H | H | D | H |
| VII-65 | D | D | H | H | H | D |
| VII-66 | D | D | H | H | D | D |
| VII-67 | D | H | D | H | D | H |
| VII-68 | D | H | D | H | H | D |
| VII-69 | D | H | D | H | D | D |
| VII-70 | D | H | H | D | D | H |
| VII-71 | D | H | H | D | H | D |
| VII-72 | D | H | H | D | D | D |
| VII-73 | D | D | D | H | D | H |
| VII-74 | D | D | D | H | H | D |
| VII-75 | D | D | D | H | D | D |
| VII-76 | D | D | H | D | D | H |
| VII-77 | D | D | H | D | H | D |
| VII-78 | D | D | H | D | D | D |
| VII-79 | D | H | D | D | D | H |
| VII-80 | D | H | D | D | H | D |
| VII-81 | D | H | D | D | D | D |
| VII-82 | D | D | D | D | D | H |
| VII-83 | D | D | D | D | H | D |
| VII-84 | D | D | D | D | D | D |

TABLE 14
Compounds of Formula VII, wherein each of R⁵ and R^{5′} = D and
each of R⁴, R^{4′}, R⁶, R^{6′} and R^{6″} = H.

| Compound # | R¹ | R^{xa} | R^{xb}/R^{xb′} | R^{xc} | R^{ya}/R^{ya′} | R^{yb}/R^{yb′} |
|---|---|---|---|---|---|---|
| VII-85 | H | D | H | H | D | H |
| VII-86 | H | D | H | H | H | D |
| VII-87 | H | D | H | H | D | D |
| VII-88 | H | H | D | H | D | H |
| VII-89 | H | H | D | H | H | D |
| VII-90 | H | H | D | H | D | D |
| VII-91 | H | H | H | D | D | H |
| VII-92 | H | H | H | D | H | D |
| VII-93 | H | H | H | D | D | D |
| VII-94 | H | D | D | H | D | H |
| VII-95 | H | D | D | H | H | D |
| VII-96 | H | D | D | H | D | D |
| VII-97 | H | D | H | D | D | H |
| VII-98 | H | D | H | D | H | D |
| VII-99 | H | D | H | D | D | D |
| VII-100 | H | H | D | D | D | H |
| VII-101 | H | H | D | D | H | D |
| VII-102 | H | H | D | D | D | D |
| VII-103 | H | D | D | D | D | H |
| VII-104 | H | D | D | D | H | D |
| VII-105 | H | D | D | D | D | D |
| VII-106 | D | D | H | H | D | H |
| VII-107 | D | D | H | H | H | D |
| VII-108 | D | D | H | H | D | D |
| VII-109 | D | H | D | H | D | H |
| VII-110 | D | H | D | H | H | D |
| VII-111 | D | H | D | H | D | D |
| VII-112 | D | H | H | D | D | H |
| VII-113 | D | H | H | D | H | D |
| VII-114 | D | H | H | D | D | D |
| VII-115 | D | D | D | H | D | H |
| VII-116 | D | D | D | H | H | D |
| VII-117 | D | D | D | H | D | D |
| VII-118 | D | D | H | D | D | H |
| VII-119 | D | D | H | D | H | D |
| VII-120 | D | D | H | D | D | D |
| VII-121 | D | H | D | D | D | H |
| VII-122 | D | H | D | D | H | D |
| VII-123 | D | H | D | D | D | D |
| VII-124 | D | D | D | D | D | H |
| VII-125 | D | D | D | D | H | D |
| VII-126 | D | D | D | D | D | D |

TABLE 15
Compounds of Formula VII, wherein each of R⁶, R^{6′} and R^{6″} =
D and each of R⁴, R^{4′}, R⁵ and R^{5′} = H.

| Compound # | R¹ | R^{xa} | R^{xb}/R^{xb′} | R^{xc} | R^{ya}/R^{ya′} | R^{yb}/R^{yb′} |
|---|---|---|---|---|---|---|
| VII-127 | H | D | H | H | D | H |
| VII-128 | H | D | H | H | H | D |
| VII-129 | H | D | H | H | D | D |
| VII-130 | H | H | D | H | D | H |
| VII-131 | H | H | D | H | H | D |
| VII-132 | H | H | D | H | D | D |
| VII-133 | H | H | H | D | D | H |
| VII-134 | H | H | H | D | H | D |
| VII-135 | H | H | H | D | D | D |
| VII-136 | H | D | D | H | D | H |
| VII-137 | H | D | D | H | H | D |
| VII-138 | H | D | D | H | D | D |
| VII-139 | H | D | H | D | D | H |
| VII-140 | H | D | H | D | H | D |
| VII-141 | H | D | H | D | D | D |
| VII-142 | H | H | D | D | D | H |
| VII-143 | H | H | D | D | H | D |
| VII-144 | H | H | D | D | D | D |
| VII-145 | H | D | D | D | D | H |
| VII-146 | H | D | D | D | H | D |
| VII-147 | H | D | D | D | D | D |
| VII-148 | D | D | H | H | D | H |
| VII-149 | D | D | H | H | H | D |
| VII-150 | D | D | H | H | D | D |
| VII-151 | D | H | D | H | D | H |
| VII-152 | D | H | D | H | H | D |
| VII-153 | D | H | D | H | D | D |
| VII-154 | D | H | H | D | D | H |
| VII-155 | D | H | H | D | H | D |
| VII-156 | D | H | H | D | D | D |
| VII-157 | D | D | D | H | D | H |
| VII-158 | D | D | D | H | H | D |
| VII-159 | D | D | D | H | D | D |
| VII-160 | D | D | H | D | D | H |
| VII-161 | D | D | H | D | H | D |
| VII-162 | D | D | H | D | D | D |
| VII-163 | D | H | D | D | D | H |
| VII-164 | D | H | D | D | H | D |
| VII-165 | D | H | D | D | D | D |
| VII-166 | D | D | D | D | D | H |
| VII-167 | D | D | D | D | H | D |
| VII-168 | D | D | D | D | D | D |

TABLE 16
Compounds of Formula VII, wherein each of R⁴, R^{4′}, R⁵ and
R^{5′} = D and each of R⁶, R^{6′} and R^{6″} = H.

| Compound # | R¹ | R^{xa} | R^{xb}/R^{xb′} | R^{xc} | R^{ya}/R^{ya′} | R^{yb}/R^{yb′} |
|---|---|---|---|---|---|---|
| VII-169 | H | D | H | H | D | H |
| VII-170 | H | D | H | H | H | D |
| VII-171 | H | D | H | H | D | D |
| VII-172 | H | H | D | H | D | H |
| VII-173 | H | H | D | H | H | D |
| VII-174 | H | H | D | H | D | D |
| VII-175 | H | H | H | D | D | H |
| VII-176 | H | H | H | D | H | D |
| VII-177 | H | H | H | D | D | D |
| VII-178 | H | D | D | H | D | H |
| VII-179 | H | D | D | H | H | D |
| VII-180 | H | D | D | H | D | D |
| VII-181 | H | D | H | D | D | H |
| VII-182 | H | D | H | D | H | D |
| VII-183 | H | D | H | D | D | D |
| VII-184 | H | H | D | D | D | H |
| VII-185 | H | H | D | D | H | D |
| VII-186 | H | H | D | D | D | D |
| VII-187 | H | D | D | D | D | H |
| VII-188 | H | D | D | D | H | D |
| VII-189 | H | D | D | D | D | D |
| VII-190 | D | D | H | H | D | H |
| VII-191 | D | D | H | H | H | D |
| VII-192 | D | D | H | H | D | D |
| VII-193 | D | H | D | H | D | H |
| VII-194 | D | H | D | H | H | D |
| VII-195 | D | H | D | H | D | D |
| VII-196 | D | H | H | D | D | H |
| VII-197 | D | H | H | D | H | D |
| VII-198 | D | H | H | D | D | D |
| VII-199 | D | D | D | H | D | H |
| VII-200 | D | D | D | H | H | D |
| VII-201 | D | D | D | H | D | D |
| VII-202 | D | D | H | D | D | H |
| VII-203 | D | D | H | D | H | D |
| VII-204 | D | D | H | D | D | D |
| VII-205 | D | H | D | D | D | H |
| VII-206 | D | H | D | D | H | D |
| VII-207 | D | H | D | D | D | D |
| VII-208 | D | D | D | D | D | H |
| VII-209 | D | D | D | D | H | D |
| VII-210 | D | D | D | D | D | D |

TABLE 17
Compounds of Formula VII, wherein each of R⁴, R^{4′}, R⁶, R^{6′} and
R^{6″} = D and each of R⁵ and R^{5′} = H.

| Compound # | R¹ | R^{xa} | R^{xb}/R^{xb′} | R^{xc} | R^{ya}/R^{ya′} | R^{yb}/R^{yb′} |
|---|---|---|---|---|---|---|
| VII-211 | H | D | H | H | D | H |
| VII-212 | H | D | H | H | H | D |
| VII-213 | H | D | H | H | D | D |
| VII-214 | H | H | D | H | D | H |
| VII-215 | H | H | D | H | H | D |
| VII-216 | H | H | D | H | D | D |
| VII-217 | H | H | H | D | D | H |
| VII-218 | H | H | H | D | H | D |
| VII-219 | H | H | H | D | D | D |
| VII-220 | H | D | D | H | D | H |
| VII-221 | H | D | D | H | H | D |
| VII-222 | H | D | D | H | D | D |
| VII-223 | H | D | H | D | D | H |
| VII-224 | H | D | H | D | H | D |
| VII-225 | H | D | H | D | D | D |
| VII-226 | H | H | D | D | D | H |
| VII-227 | H | H | D | D | H | D |
| VII-228 | H | H | D | D | D | D |
| VII-229 | H | D | D | D | D | H |
| VII-230 | H | D | D | D | H | D |
| VII-231 | H | D | D | D | D | D |
| VII-232 | D | D | H | H | D | H |
| VII-233 | D | D | H | H | H | D |
| VII-234 | D | D | H | H | D | D |
| VII-235 | D | H | D | H | D | H |
| VII-236 | D | H | D | H | H | D |
| VII-237 | D | H | D | H | D | D |
| VII-238 | D | H | H | D | D | H |
| VII-239 | D | H | H | D | H | D |
| VII-240 | D | H | H | D | D | D |
| VII-241 | D | D | D | H | D | H |
| VII-242 | D | D | D | H | H | D |
| VII-243 | D | D | D | H | D | D |
| VII-244 | D | D | H | D | D | H |
| VII-245 | D | D | H | D | H | D |
| VII-246 | D | D | H | D | D | D |
| VII-247 | D | H | D | D | D | H |
| VII-248 | D | H | D | D | H | D |
| VII-249 | D | H | D | D | D | D |
| VII-250 | D | D | D | D | D | H |
| VII-251 | D | D | D | D | H | D |
| VII-252 | D | D | D | D | D | D |

TABLE 18
Compounds of Formula VII,
wherein each of R⁵, R^{5′}, R⁶, R^{6′} and R^{6″} = D and each of R⁴ and R^{4′} = H.

| Compound # | R¹ | R^{xa} | R^{xb}/R^{xb′} | R^{xc} | R^{ya}/R^{ya′} | R^{yb}/R^{yb′} |
|---|---|---|---|---|---|---|
| VII-253 | H | D | H | H | D | H |
| VII-254 | H | D | H | H | H | D |
| VII-255 | H | D | H | H | D | D |
| VII-256 | H | H | D | H | D | H |
| VII-257 | H | H | D | H | H | D |
| VII-258 | H | H | D | H | D | D |
| VII-259 | H | H | H | D | D | H |
| VII-260 | H | H | H | D | H | D |
| VII-261 | H | H | H | D | D | D |
| VII-262 | H | D | D | H | D | H |
| VII-263 | H | D | D | H | H | D |
| VII-264 | H | D | D | H | D | D |
| VII-265 | H | D | H | D | D | H |
| VII-266 | H | D | H | D | H | D |
| VII-267 | H | D | H | D | D | D |
| VII-268 | H | H | D | D | D | H |
| VII-269 | H | H | D | D | H | D |
| VII-270 | H | H | D | D | D | D |
| VII-271 | H | D | D | D | D | H |
| VII-272 | H | D | D | D | H | D |
| VII-273 | H | D | D | D | D | D |
| VII-274 | D | D | H | H | D | H |
| VII-275 | D | D | H | H | H | D |
| VII-276 | D | D | H | H | D | D |
| VII-277 | D | H | D | H | D | H |
| VII-278 | D | H | D | H | H | D |
| VII-279 | D | H | D | H | D | D |
| VII-280 | D | H | H | D | D | H |
| VII-281 | D | H | H | D | H | D |
| VII-282 | D | H | H | D | D | D |
| VII-283 | D | D | D | H | D | H |
| VII-284 | D | D | D | H | H | D |
| VII-285 | D | D | D | H | D | D |
| VII-286 | D | D | H | D | D | H |
| VII-287 | D | D | H | D | H | D |
| VII-288 | D | D | H | D | D | D |
| VII-289 | D | H | D | D | D | H |
| VII-290 | D | H | D | D | H | D |
| VII-291 | D | H | D | D | D | D |
| VII-292 | D | D | D | D | D | H |
| VII-293 | D | D | D | D | H | D |
| VII-294 | D | D | D | D | D | D |

TABLE 19
Compounds of Formula VII, wherein each of
R⁴, R^{4′}, R⁵, R^{5′}, R⁶, R^{6′} and R^{6″} = D.

| Compound # | R¹ | R^{xa} | R^{xb}/R^{xb′} | R^{xc} | R^{ya}/R^{ya′} | R^{yb}/R^{yb′} |
|---|---|---|---|---|---|---|
| VII-295 | H | D | H | H | D | H |
| VII-296 | H | D | H | H | H | D |
| VII-297 | H | D | H | H | D | D |
| VII-298 | H | H | D | H | D | H |
| VII-299 | H | H | D | H | H | D |
| VII-300 | H | H | D | H | D | D |
| VII-301 | H | H | H | D | D | H |
| VII-302 | H | H | H | D | H | D |
| VII-303 | H | H | H | D | D | D |
| VII-304 | H | D | D | H | D | H |
| VII-305 | H | D | D | H | H | D |
| VII-306 | H | D | D | H | D | D |
| VII-307 | H | D | H | D | D | H |
| VII-308 | H | D | H | D | H | D |
| VII-309 | H | D | H | D | D | D |
| VII-310 | H | H | D | D | D | H |
| VII-311 | H | H | D | D | H | D |
| VII-312 | H | H | D | D | D | D |
| VII-313 | H | D | D | D | D | H |
| VII-314 | H | D | D | D | H | D |
| VII-315 | H | D | D | D | D | D |
| VII-316 | D | D | H | H | D | H |
| VII-317 | D | D | H | H | H | D |
| VII-318 | D | D | H | H | D | D |
| VII-319 | D | H | D | H | D | H |
| VII-320 | D | H | D | H | H | D |
| VII-321 | D | H | D | H | D | D |
| VII-322 | D | H | H | D | D | H |
| VII-323 | D | H | H | D | H | D |
| VII-324 | D | H | H | D | D | D |
| VII-325 | D | D | D | H | D | H |
| VII-326 | D | D | D | H | H | D |
| VII-327 | D | D | D | H | D | D |
| VII-328 | D | D | H | D | D | H |
| VII-329 | D | D | H | D | H | D |
| VII-330 | D | D | H | D | D | D |
| VII-331 | D | H | D | D | D | H |
| VII-332 | D | H | D | D | H | D |
| VII-333 | D | H | D | D | D | D |
| VII-334 | D | D | D | D | D | H |
| VII-335 | D | D | D | D | H | D |
| VII-336 | D | D | D | D | D | D |

In some embodiments, the present invention provides compounds comprising one, two, three, four, five, six, seven, eight, nine, ten, eleven, twelve, thirteen, fourteen, fifteen, sixteen, seventeen, eighteen or nineteen deuterium atoms. In some embodiments, provided compounds comprise deuterium in an amount of about 5%, or about 10%, or about 15%, or about 21%, or about 26%, or about 31%, or about 36%, or about 42%, or about 47%, or about 52%, or about 57%, or about 63%, or about 68%, or about 73%, or about 78%, or about 84%, or about 89%, or about 94%, or about 100%. In some embodiments, the present invention provides deuterium-enriched compounds according to Table 20:

TABLE 20
Deuterium Enriched Compounds
of Formula I.

| Number of | |
|---|---|
| Deuterium | Percent |
| Atoms | Enrichment |
| 1 | 5.3 |
| 2 | 10.5 |
| 3 | 15.8 |
| 4 | 21.1 |
| 5 | 26.3 |
| 6 | 31.6 |
| 7 | 36.8 |
| 8 | 42.1 |
| 9 | 47.4 |
| 10 | 52.6 |
| 11 | 57.9 |
| 12 | 63.2 |
| 13 | 68.4 |
| 14 | 73.7 |
| 15 | 78.9 |
| 16 | 84.2 |
| 17 | 89.5 |
| 18 | 94.7 |
| 19 | 100 |

In some embodiments, the present invention provides a compound selected from any of Tables 1-19 above, or a pharmaceutically acceptable salt thereof.

In some embodiments, the present invention provides an isolated or purified compound of any of Formulae I, II, III, IV, V, VI or VII.

Compounds of Formulae I, II, III, IV, V, VI or VII contain an acrylamide moiety, which is capable of and particularly suitable for covalently binding to a key cysteine residue in the binding domain of certain protein kinases. Protein kinases having a cysteine residue in the binding domain are known to one of ordinary skill in the art and include ErbB1, ErbB2, and ErbB4, or a mutant thereof. In certain embodiments, compounds of the present invention having an acrylamide group target one or more of the following cysteine residues:

| | |
|---|---|
| ERBB1 | ITQLMPFGCLLDYVREH |
| ERBB2 | VTQLMPYGCLLDHVREN |
| ERBB4 | VTQLMPHGCLLEYVHEH |

Thus, in some embodiments, compounds of the present invention having an acrylamide group are capable of covalently binding to a cysteine residue thereby irreversibly inhibiting the enzyme. In certain embodiments, the cysteine residue is Cys797 of ErbB1, Cys805 of ErbB2 and Cys803 of ErbB4, or a mutant thereof, where the provided residue numbering is in accordance with Uniprot (code POO533 for ErbB1; code PO4626 for ErbB2, and Q15303 for ErbB4). It will be understood that the Cys of ErbB1 (EGFR) is variably called 773 or 797 depending on whether the parent sequence contains the signal peptide or not. Thus, in accordance with the present invention, the relevant cysteine residue of ErbB1 may be described as Cys 773 or Cys 797 and these terms are used interchangeably.

In certain embodiments, compounds of the present invention having an acrylamide group are capable of covalently binding to a cysteine residue of TEC, thereby irreversibly inhibiting the enzyme. In some embodiments, the cysteine residue is Cys 449.

In certain embodiments, compounds of the present invention having an acrylamide group are capable of covalently binding to a cysteine residue of BTK, thereby irreversibly inhibiting the enzyme. In some embodiments, the cysteine residue is Cys 481.

In certain embodiments, compounds of the present invention having an acrylamide group are capable of covalently binding to a cysteine residue of ITK, thereby irreversibly inhibiting the enzyme. In some embodiments, the cysteine residue is Cys 442.

In certain embodiments, compounds of the present invention having an acrylamide group are capable of covalently binding to a cysteine residue of BMX, thereby irreversibly inhibiting the enzyme. In some embodiments, the cysteine residue is Cys 496.

In certain embodiments, compounds of the present invention having an acrylamide group are capable of covalently binding to a cysteine residue of JAK3, thereby irreversibly inhibiting the enzyme. In some embodiments, the cysteine residue is Cys 909.

In certain embodiments, compounds of the present invention having an acrylamide group are capable of covalently binding to a cysteine residue of TXK, thereby irreversibly inhibiting the enzyme. In some embodiments, the cysteine residue is Cys 350.

As described herein, certain compounds of the present invention are irreversible inhibitors of at least one of ErbB1, ErbB2, ErbB3 and ErbB4, or a mutant thereof. In some embodiments, provided compounds are irreversible inhibitors of a TEC-kinase (e.g., TEC, BTK, ITK, RLK or BMX) and JAK3, or a mutant thereof, and therefore useful for treating one or disorders as described herein.

### 4. Uses, Formulation and Administration

### Pharmaceutically Acceptable Compositions

According to another embodiment, the invention provides a composition comprising a compound of this invention or a pharmaceutically acceptable derivative thereof and a pharmaceutically acceptable carrier, adjuvant, or vehicle. The amount of compound in compositions of this invention is such that is effective to measurably inhibit a protein kinase, particularly at least one of ErbB1, ErbB2, ErbB3, ErbB4, a TEC-kinase (e.g., TEC, BTK, ITK, RLK or BMX), and/or JAK3, or a mutant thereof, in a biological sample or in a patient. In certain embodiments, the amount of compound in compositions of this invention is such that is effective to measurably inhibit at least one of ErbB1, ErbB2, ErbB3, ErbB4, a TEC-kinase (e.g., TEC, BTK, ITK, RLK or BMX), and/or JAK3, or a mutant thereof, in a biological sample or in a patient. In certain embodiments, a composition of this invention is formulated for administration to a patient in need of such composition. In some embodiments, a composition of this invention is formulated for oral administration to a patient.

In some embodiments, the present invention provides a pharmaceutical composition comprising a compound of any of Formulae I, II, III, IV, V, VI or VII, or a pharmaceutically acceptable salt thereof, and one or more pharmaceutically acceptable excipients.

The term “patient”, as used herein, means an animal, preferably a mammal, and most preferably a human.

The term “pharmaceutically acceptable carrier, adjuvant, vehicle or excipient” refers to a non-toxic carrier, adjuvant, vehicle or excipient that does not destroy the pharmacological activity of the compound with which it is formulated. Pharmaceutically acceptable carriers, adjuvants, vehicles or excipients that may be used in the compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

A “pharmaceutically acceptable derivative” means any non-toxic salt, ester, salt of an ester or other derivative of a compound of this invention that, upon administration to a recipient, is capable of providing, either directly or indirectly, a compound of this invention or an inhibitorily active metabolite or residue thereof.

As used herein, the term “inhibitorily active metabolite or residue thereof” means that a metabolite or residue thereof is also an inhibitor of at least one of ErbB1, ErbB2, ErbB3, ErbB4, a TEC-kinase (e.g., TEC, BTK, ITK, RLK or BMX), and/or JAK3, or a mutant thereof.

Compositions of the present invention may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. Preferably, the compositions are administered orally, intraperitoneally or intravenously. Sterile injectable forms of the compositions of this invention may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

Pharmaceutically acceptable compositions of this invention may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added.

Alternatively, pharmaceutically acceptable compositions of this invention may be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols.

Pharmaceutically acceptable compositions of this invention may also be administered topically, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract can be effected in a rectal suppository formulation (see above) or in a suitable enema formulation. Topically-transdermal patches may also be used.

For topical applications, provided pharmaceutically acceptable compositions may be formulated in a suitable ointment containing the active component suspended or dissolved in one or more carriers. Carriers for topical administration of compounds of this invention include, but are not limited to, mineral oil, liquid petrolatum, white petrolatum, propylene glycol, polyoxyethylene, polyoxypropylene compound, emulsifying wax and water. Alternatively, provided pharmaceutically acceptable compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

For ophthalmic use, provided pharmaceutically acceptable compositions may be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic uses, the pharmaceutically acceptable compositions may be formulated in an ointment such as petrolatum.

Pharmaceutically acceptable compositions of this invention may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

Most preferably, pharmaceutically acceptable compositions of this invention are formulated for oral administration.

The amount of compounds of the present invention that may be combined with the carrier materials to produce a composition in a single dosage form will vary depending upon the host treated, the particular mode of administration. Preferably, provided compositions should be formulated so that a dosage of between 0.01-100 mg/kg body weight/day of the inhibitor can be administered to a patient receiving these compositions.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of a compound of the present invention in the composition will also depend upon the particular compound in the composition.

### Uses of Compounds and Pharmaceutically Acceptable Compositions

Compounds and compositions described herein are generally useful for the inhibition of protein kinase activity of one or more enzymes.

Drug resistance is emerging as a significant challenge for targeted therapies. For example, drug resistance has been reported for Gleevec® and Iressa®, as well as several other kinase inhibitors in development. In addition, drug resistance has been reported for the cKit and PDGFR receptors. It has been reported that irreversible inhibitors may be effective against drug resistant forms of protein kinases (Kwak, E. L., R. Sordella, et al. (2005). “Irreversible inhibitors of the EGF receptor may circumvent acquired resistance to gefitinib.” PNAS 102(21): 7665-7670.) Without wishing to be bound by any particular theory, it is believed that compounds of the present invention may be effective inhibitors of drug resistant forms of protein kinases.

As used herein, the term “clinical drug resistance” refers to the loss of susceptibility of a drug target to drug treatment as a consequence of mutations in the drug target.

As used herein, the term “resistance” refers to changes in the wild-type nucleic acid sequence coding a target protein, and/or the protein sequence of the target, which changes decrease or abolish the inhibitory effect of the inhibitor on the target protein.

Examples of kinases that are inhibited by the compounds and compositions described herein and against which the methods described herein are useful include ErbB1, ErbB2, ErbB3, ErbB4, a TEC-kinase (including TEC, BTK, ITK, RLK or BMX), and/or JAK3, or a mutant thereof.

The activity of a compound utilized in this invention as an inhibitor of ErbB1, ErbB2, ErbB3, ErbB4, a TEC-kinase (e.g., TEC, BTK, ITK, RLK or BMX), and/or JAK3, or a mutant thereof, may be assayed in vitro, in vivo or in a cell line. In vitro assays include assays that determine inhibition of either the phosphorylation activity and/or the subsequent functional consequences, or ATPase activity of activated ErbB1, ErbB2, ErbB3, ErbB4, a TEC-kinase (e.g., TEC, BTK, ITK, RLK or BMX), and/or JAK3, or a mutant thereof. Alternate in vitro assays quantitate the ability of the inhibitor to bind to ErbB1, ErbB2, ErbB3, ErbB4, a TEC-kinase (e.g., TEC, BTK, ITK, RLK or BMX), and/or JAK3. Inhibitor binding may be measured by radiolabeling the inhibitor prior to binding, isolating the inhibitor/ErbB1, inhibitor/ErbB2, inhibitor/ErbB3, inhibitor/ErbB4, inhibitor/TEC-kinase (e.g., TEC, BTK, ITK, RLK or BMX), or inhibitor/JAK3 complex and determining the amount of radiolabel bound. Alternatively, inhibitor binding may be determined by running a competition experiment where new inhibitors are incubated with ErbB1, ErbB2, ErbB3, ErbB4, a TEC-kinase (e.g., TEC, BTK, ITK, RLK or BMX), and/or JAK3 bound to known radioligands. Detailed conditions for assaying a compound utilized in this invention as an inhibitor of ErbB1, ErbB2, ErbB3, ErbB4, a TEC-kinase (e.g., TEC, BTK, ITK, RLK or BMX), and/or JAK3, or a mutant thereof, are set forth in the Examples below.

Protein tyrosine kinases are a class of enzymes that catalyze the transfer of a phosphate group from ATP or GTP to a tyrosine residue located on a protein substrate. Receptor tyrosine kinases act to transmit signals from the outside of a cell to the inside by activating secondary messaging effectors via a phosphorylation event. A variety of cellular processes are promoted by these signals, including proliferation, carbohydrate utilization, protein synthesis, angiogenesis, cell growth, and cell survival.

### (a) ErbB Family

ErbB receptors, a major family of receptor tyrosine kinases, are composed of an extracellular ligand binding domain, a single transmembrane domain, and an intracellular domain with tyrosine kinase activity. The ErbB family comprises ErbB1 (commonly known as EGFR), ErbB2 (commonly known as HER2 or neu), ErbB3 (commonly known as HER3), and ErbB4 (commonly known as HER4). More than 10 ligands (including EGF, TGFα, AR, BTC, EPR, HB-EGF, NRG-1, NRG-2, NRG-3, NRG-4) have been identified for the various receptor family members. Upon ligand binding the extracellular domain undergoes conformational change, allowing the formation of homodimers or heterodimers with other members of the ErbB family. Dimerization induces tyrosine phosphorylation of specific residues in the intracellular domain that serve as docking sites for adaptor proteins and downstream effectors. In some contexts, activation of phosphatidyl-inositol 3-kinase (PI3K) and mitogen-activated protein kinase pathways occur, leading to cell proliferation and survival (Lin, N. U.; Winer, E. P., Breast Cancer Res 6: 204-210, 2004).

Interaction between family members is necessitated by deficiencies in ErbB2, which has no known ligand, and ErbB3, which is kinase dead. EGFR, ErbB3, and ErbB4 bind ligand to induce ErbB receptor homodimerization or heterodimerization, whereas ErbB2 functions as the preferred dimerization partner. The composition of the pairwise combinations is important for signal diversification, as dimer identity determines which downstream pathways are activated. Representative downstream gene products in the ErbB signal transduction pathway include Shc, Grb2, SOS1, Ras, Raf1, Mek, ERK1, ERK2, ERα, Akt, mTOR, FKHR, p27, Cyclin D1, FasL, GSK-3, Bad, and STAT3.

There is strong precedent for involvement of the EGFR and other members of the ErbB family in human cancer because over 60% of all solid tumors overexpress at least one of these proteins or their ligands. Constitutively active, tumorigenic EGFR vIII, a mutant possessing a truncated extracellular domain, has been reported to be present in up to 78% of breast carcinomas and has also been found in glioblastomas. Overexpression of EGFR is commonly found in breast, lung, head and neck, bladder tumors, while ErbB2 expression is frequently elevated in human tumors of epithelial origin. Activating mutations in the tyrosine kinase domain have been identified in patients with non-small cell lung cancer (Lin, N. U.; Winer, E. P., Breast Cancer Res 6: 204-210, 2004). ErbB1 and/or ErbB2 amplification has also been implicated in squamous cell carcinomas, salivary gland carcinomas, ovarian carcinomas, and pancreatic cancers (Cooper, G. C. Oncogenes. 2^{nd} ed. Sudbury: Jones and Barlett, 1995; Zhang, Y., et al., Cancer Res 66: 1025-32, 2006). Overexpression of ErbB2 has potent transforming activity, likely due to its ability to cooperate with other ErbB receptors (Sherman, L., et al., Oncogene 18: 6692-99, 1999). In fact, some human cancers that overexpress both EGFR and ErbB2 have a poorer prognosis than cancers that overexpress either receptor alone.

The ErbB signaling network is often a key component in the pathogenesis of breast cancer. Amplification of ErbB2 is associated with an aggressive tumor phenotype that is characterized by relatively rapid tumor growth, metastatic spread to visceral sites, and drug resistance. ErbB2 has been shown to be amplified in 20% of axillary node-negative (“ANN”) breast cancer cases, and this amplification has been identified as an independent prognostic factor for risk of recurrence in ANN breast cancer. (Andrulis, I. L., et al., J Clin Oncol 16: 1340-9, 1998).

Targeted blockade of ErbB signaling with trastuzumab (Herceptin), a monoclonal antibody directed at ErbB2, has been shown to improve survival in women with ErbB2-positive, advanced breast cancer. Other monoclonal antibodies directed against ErbB receptors include cetuximab (Erbitux) and panitumumab (Vectibix).

Several small molecule tyrosine kinase inhibitors (TKIs) have been found to act selectively upon ErbB family members. Notable examples include gefitinib (Iressa) and erlotinib (Tarceva), both of which target the EGFR. These small molecules compete with ATP for binding to the kinase domain of the receptor. Compared to monoclonal antibodies, TKIs have several advantages in that they are orally bioavailable, well-tolerated, and appear to be active against truncated forms of ErbB2 and EGFR receptors (e.g., EGFR vIII) in vitro. In addition, the small size of small molecule TKIs may allow them to penetrate sanctuary sites such as the central nervous system. Finally, the homology between kinase domains of ErbB receptors allows for development of TKIs that target more than one member of the ErbB family simultaneously, the advantages of which are described herein.

Although certain malignancies have been linked to the overexpression of individual receptors, efficient signal transduction relies on the coexpression of ErbB receptor family members. This cooperation of ErbB receptor family members in signal transduction and malignant transformation may limit the success of agents that target individual receptors in the treatment of cancer; a potential mechanism of resistance to agents targeting a single ErbB receptor is upregulation of other members of the receptor family (Britten, C. D., Mol Cancer Ther 3: 1335-42, 2004).

Agents that target two or more ErbB receptors are called pan-ErbB regulators. ERRP is a pan-ErbB negative regulator that is expressed in most benign pancreatic ductal epithelium and islet cells. Tumors have been found to experience a progressive loss in ERRP expression. That Erbitux and Herceptin show success in a limited patient base (tumors having increased expression of EGFR or ErbB2) could be partly due to coexpression of multiple ErbB family members.

In both in vitro and in vivo models, strategies that employ a dual ErbB approach seem to have greater antitumor activity than agents targeting a single ErbB receptor. Thus, agents that target multiple members of ErbB family are likely to provide therapeutic benefit to a broader patient population (Zhang, Y., et al., Cancer Res 66: 1025-32, 2006). In certain embodiments, provided compounds inhibit one or more of ErbB1, ErbB2, ErbB3, and ErbB4. In some embodiments, provided compounds inhibit two or more of ErbB1, ErbB2, ErbB3, and ErbB4, or a mutant thereof, and are therefore pan-ErbB inhibitors.

Clearly, there is growing evidence to support the concurrent inhibition of two or more ErbB (e.g., pan-ErbB) receptors in cancer therapy. Possible pan-ErbB approaches with small molecules include using combinations of agents that target individual ErbB receptors, using single agents that target multiple ErbB receptors, or using agents that interfere with ErbB receptor interactions (e.g., dimerization). Additional strategies include therapies utilizing a small molecule in combination with antibodies, or chemoprevention therapies (Lin, N. U.; Winer, E. P., Breast Cancer Res 6: 204-210, 2004).

An example of small molecule pan-ErbB inhibition is CI-1033, an irreversible pan-ErbB inhibitor that covalently binds to the ATP binding site of the intracellular kinase domain. Another irreversible pan-ErbB receptor tyrosine kinase inhibitor is HKI-272, which inhibits the growth of tumor cells that express ErbB-1 (EGFR) and ErbB-2 (HER-2) in culture and xenografts, and has antitumor activity in HER-2-positive breast cancer (Andrulis, I. L., et al., J Clin Oncol 16: 1340-9, 1998). Irreversible inhibitors have demonstrated superior antitumor activity in comparison with reversible inhibitors.

Neurofibromatosis type I (NF1) is a dominantly inherited human disease affecting one in 2500-3500 individuals. Several organ systems are affected, including bones, skin, iris, and the central nervous system, as manifested in learning disabilities and gliomas. A hallmark of NF1 is the development of benign tumors of the peripheral nervous system (neurofibromas), which vary greatly in both number and size among patients. Neurofibromas are heterogeneous tumors composed of Schwann cells, neurons, fibroblasts and other cells, w/Schwann cells being the major (60-80%) cell type.

Abberant expression of the EGFR is associated with tumor development in NF1 and in animal models of NF1, suggesting a role in pathogenesis and representing a novel potential therapeutic target. EGFR expression affects the growth of tumor cell lines derived from NF1 patients under conditions where EGF is not the primary factor driving growth of the cells. These data suggest that EGFR may play an important role in NF1 tumorigenesis and Schwann cell transformation (DeClue, J. E., et al., J Clin Invest 105: 1233-41, 2000).

Patients with NF1 develop aggressive Schwann cell neoplasmas known as malignant peripheral nerve sheath tumors (MPNSTs). Schwann cells are the major supportive cell population in the peripheral nervous system. Neoplastic Schwann cells within these neoplasms variably express the ErbB tyrosine kinases mediating NRG-1 responses (ErbB2, ErbB3, ErbB4). Neuregulin-1 (NRG-1) proteins promote the differentiation, survival, and/or proliferation of many cell types in the developing nervous system, and overexpression of NRG-1 in myelinating Schwann cells induces the formation of malignant peripheral nerve sheath tumors (MPNSTs) (Fallon, K. B., et al., J Neuro Oncol 66: 273-84, 2004).

Deregulation of Schwann cell growth is a primary defect driving the development of both benign neurofibromas and MPNST in neurofibromatosis type I (NF1) patients. Growth of MPNSTs and transformed mouse Schwann cells in vitro is highly EGF-dependent and can be blocked by EGFR inhibitors under conditions where EGF is the primary growth factor. Some human MPNST cell lines have been found to demonstrate constitutive ErbB phosphorylation. While treatment with ErbB inhibitors abolishes ErbB phosphorylation and reduces DNA synthesis in these lines, effective chemotherapeutic regimens for MPNST remain elusive (Stonecypher, M. S., et al., Oncogene 24: 5589-5605, 2005).

Schwannomas are peripheral nerve tumors comprised almost entirely of Schwann-like cells, and typically have mutations in the neurofibromatosis type II (NF2) tumor suppressor gene. Ninety percent of NF2 patients develop bilateral vestibular schwannomas and/or spinal schwannomas. Enlarging schwannomas can compress adjacent structures, resulting in deafness and other neurologic problems. Surgical removal of these tumors is difficult, often resulting in increased patient morbidity.

Both normal human Schwann cells and schwannoma cells express neuregulin receptors (e.g., ErbB receptors), and schwannoma cells proliferate in response to neuregulin. It is possible that aberrant neuregulin production or response contributes to aberrant schwannoma cell proliferation (Pelton, P. D., et al., Oncogene 17: 2195-2209, 1998).

The NF2 tumor suppressor, Merlin, is a membrane/cytoskeleton-associated protein implicated in the regulation of tyrosine kinase activity. Genetic interactions between a Merlin mutation and EGFR pathway mutations have been documented in Drosophila (LaJeunesse, D. R., et al., Genetics 158: 667-79, 2001). Other evidence suggests Merlin can inhibit EGFR internalization and signaling upon cell-cell contact by restraining the EGFR into a membrane compartment from which it can neither signal nor be internalized (McClatchey, A. I., et al., Genes and Development 19: 2265-77, 2005; Curto, M. C., et al., J Cell Biol 177: 893-903, 2007).

As used herein, the terms “treatment,” “treat,” and “treating” refer to reversing, alleviating, delaying the onset of, or inhibiting the progress of a disease or disorder, or one or more symptoms thereof, as described herein. In some embodiments, treatment may be administered after one or more symptoms have developed. In other embodiments, treatment may be administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved, for example to prevent or delay their recurrence.

Provided compounds are inhibitors of one or more of ErbB1, ErbB2, ErbB3, and ErbB4 and are therefore useful for treating one or more disorders associated with activity of one of more of ErbB1, ErbB2, ErbB3, and ErbB4. Thus, in certain embodiments, the present invention provides a method for treating an ErbB1-mediated, an ErbB2-mediated, an ErbB3-mediated, and/or ErbB4-mediated disorder comprising the step of administering to a patient in need thereof a compound of the present invention, or pharmaceutically acceptable composition thereof.

As used herein, the terms “ErbB1-mediated”, “ErbB2-mediated,” “ErbB3-mediated,” and/or “ErbB4-mediated” disorders or conditions as used herein means any disease or other deleterious condition in which one or more of ErbB1, ErbB2, ErbB3, and/or ErbB4, or a mutant thereof, are known or suspected to play a role. Accordingly, another embodiment of the present invention relates to treating or lessening the severity of one or more diseases in which one or more of ErbB1, ErbB2, ErbB3, and/or ErbB4, or a mutant thereof, are known or suspected to play a role. Specifically, the present invention relates to a method of treating or lessening the severity of a disease or condition selected from a proliferative disorder, wherein said method comprises administering to a patient in need thereof a compound or composition according to the present invention.

In some embodiments, the present invention provides a method for treating or lessening the severity of one or more disorders selected from a cancer. In some embodiments, the cancer is associated with a solid tumor. In certain embodiments, the cancer is breast cancer, glioblastoma, lung cancer, cancer of the head and neck, colorectal cancer, bladder cancer, or non-small cell lung cancer. In some embodiments, the present invention provides a method for treating or lessening the severity of one or more disorders selected from squamous cell carcinoma, salivary gland carcinoma, ovarian carcinoma, or pancreatic cancer.

In certain embodiments, the present invention provides a method for treating or lessening the severity of neurofibromatosis type I (NF1), neurofibromatosis type II (NF2) Schwann cell neoplasms (e.g. MPNST's), or Schwannomas.

### (b) TEC Family

The TEC family of non-receptor tyrosine kinases, referred to herein as “TEC-kinases,” plays a central role in signaling through antigen-receptors such as the TCR, BCR and Fc receptors (reviewed in Miller A, et al. Current Opinion in Immunology 14; 331-340 (2002). TEC-kinases are essential for T cell activation. Three members of the family, Itk, Rlk and Btk, are activated downstream of antigen receptor engagement in T cells and transmit signals to downstream effectors, including PLC-γ. Combined deletion of Itk and Rlk in mice leads to a profound inhibition of TCR responses including proliferation, cytokine production and immune responses to an intracellular parasite (Toxoplasma gondii) (Schaeffer et al., Science 284; 638-641 (1999)). Intracellular signalling following TCR engagement is effected in ITK/RLK deficient T cells; inositol triphosphate production, calcium mobilization and MAP kinase activation are all reduced. Tec-kinases are also essential for B cell development and activation.

TEC-kinases include five family members, which are expressed primarily in hematopoietic cells: TEC, BTK, ITK (also known as TSK and EMT), RLK (also known as TXK), and BMX (also known as ETK). Additional related TEC-kinases have been found in Drosophila melanogaster, zebrafish (Danio rerió), skate (Raja eglanteria), and sea urchin (Anthocidaris crassispina).

Provided compounds are inhibitors of one of more TEC-kinases (e.g., TEC, BTK, ITK, RLK or BMX) and are therefore useful for treating one or more disorders associated with activity of one or more TEC-kinases (e.g., TEC, BTK, ITK, RLK or BMX). Thus, in certain embodiments, the present invention provides a method for treating a TEC-mediated disorder comprising the step of administering to a patient in need thereof a compound of the present invention, or pharmaceutically acceptable composition thereof.

The term “TEC-mediated condition”, as used herein means any disease or other deleterious condition in which TEC-kinases (e.g., TEC, BTK, ITK, RLK or BMX) are known or suspected to play a role. Such conditions include those described herein and in Melcher, M et al., “The Role of TEC Family Kinases in Inflammatory Processes”, Anti-Inflammatory & Anti-Allergy Agents in Medicinal Chemistry, Vol. 6, No. 1, pp. 61-69 (February 2007). Accordingly, another embodiment of the present invention relates to treating or lessening the severity of one or more diseases in which TEC-kinases (e.g., TEC, BTK, ITK, RLK or BMX) are known or suspected to play a role. Specifically, the present invention relates to a method of treating or lessening the severity of a disease or condition selected from autoimmune, inflammatory, proliferative, and hyperproliferative diseases and immunologically-mediated diseases including rejection of transplanted organs or tissues and Acquired Immunodeficiency Syndrome (AIDS)(also known as HIV), wherein said method comprises administering to a patient in need thereof a composition of the present invention.

In some embodiments, the present invention provides a method for treating or lessening the severity of one or more diseases and conditions associated with TEC-kinases (e.g., TEC, BTK, ITK, RLK or BMX) including diseases of the respiratory tract including, without limitation, reversible obstructive airways diseases including asthma, such as bronchial, allergic, intrinsic, extrinsic and dust asthma, particularly chronic or inveterate asthma (e.g., late asthma airways hyper-responsiveness) and bronchitis. In some embodiments, the present invention provides a method for treating or lessening the severity of one or more diseases and conditions associated with TEC-kinases (e.g., TEC, BTK, ITK, RLK or BMX) including those conditions characterized by inflammation of the nasal mucus membrane, including acute rhinitis, allergic, atrophic rhinitis and chronic rhinitis including rhinitis caseosa, hypertrophic rhinitis, rhinitis purulenta, rhinitis sicca and rhinitis medicamentosa; membranous rhinitis including croupous, fibrinous and pseudomembranous rhinitis and scrofoulous rhinitis, seasonal rhinitis including rhinitis nervosa (hay fever) and vasomotor rhinitis, sarcoidosis, farmer's lung and related diseases, fibroid lung, and idiopathic interstitial pneumonia.

In some embodiments, the present invention provides a method for treating or lessening the severity of one or more diseases and conditions associated with TEC-kinases (e.g., TEC, BTK, ITK, RLK or BMX) including diseases of the bone and joints including, without limitation, rheumatoid arthritis, seronegative spondyloarthropathies (including ankylosing spondylitis, psoriatic arthritis and Reiter's disease), Behcet's disease, Sjogren's syndrome, systemic sclerosis, osteoporosis, bone cancer, and bone metastasis.

In some embodiments, the present invention provides a method for treating or lessening the severity of one or more diseases and conditions associated with TEC-kinases (i.e., TEC, BTK, ITK, RLK or BMX) including diseases and disorders of the skin, including, without limitation, psoriasis, systemic sclerosis, atopical dermatitis, contact dermatitis and other eczematous dermatitis, seborrhoetic dermatitis, Lichen planus, pemphigus, bullous pemphigus, epidermolysis bullosa, urticaria, angiodermas, vasculitides, erythemas, cutaneous eosinophilias, uveitis, alopecia, areata and vernal conjunctivitis.

In some embodiments, the present invention provides a method for treating or lessening the severity of one or more diseases and conditions associated with TEC-kinases (e.g., TEC, BTK, ITK, RLK or BMX) including diseases and disorders of the gastrointestinal tract, including, without limitation, celiac disease, proctitis, eosinophilic gastro-enteritis, mastocytosis, pancreatitis, Crohn's disease, ulcerative colitis, food-related allergies which have effects remote from the gut, e. g. migraine, rhinitis and eczema.

In some embodiments, the present invention provides a method for treating or lessening the severity of one or more diseases and conditions associated with TEC-kinases (e.g., TEC, BTK, ITK, RLK or BMX) including those diseases and disorders of other tissues and systemic disease, including, without limitation, multiple sclerosis, artherosclerosis, lupus erythematosus, systemic lupus erythematosus, Hashimoto's thyroiditis, myasthenia gravis, type I diabetes, nephrotic syndrome, eosinophilia fascitis, hyper IgE syndrome, lepromatous leprosy, sezary syndrome and idiopathic thrombocytopenia purpura, restenosis following angioplasty, tumours (for example leukemia, lymphomas, and prostate cancers), and artherosclerosis.

In some embodiments, the present invention provides a method for treating or lessening the severity of one or more diseases and conditions associated with TEC-kinases (e.g., TEC, BTK, ITK, RLK or BMX) including allograft rejection including, without limitation, acute and chronic allograft rejection following for example transplantation of kidney, heart, liver, lung, bone marrow, skin and cornea; and chronic graft versus host disease.

In some embodiments, the present invention relates to a method of treating or lessening the severity of one or more of the diseases or conditions associated with TEC-kinases (e.g., TEC, BTK, ITK, RLK or BMX), as recited above, wherein said method comprises administering to a patient in need thereof a compound or composition according to the present invention.

### (c) Bruton's Tyrosine Kinase (BTK)

Bruton's tyrosine kinase (“BTK”), a member of the TEC-kinases (e.g., TEC, BTK, ITK, RLK or BMX), is a key signaling enzyme expressed in all hematopoietic cell types except T lymphocytes and natural killer cells. BTK plays an essential role in the B-cell signaling pathway linking cell surface B-cell receptor (BCR) stimulation to downstream intracellular responses.

BTK is a key regulator of B-cell development, activation, signaling, and survival (Kurosaki, Curr Op Imm, 2000, 276-281; Schaeffer and Schwartzberg, Curr Op Imm 2000, 282-288). In addition, BTK plays a role in a number of other hematopoietic cell signaling pathways, e.g., Toll like receptor (TLR) and cytokine receptor-mediated TNF-α production in macrophages, IgE receptor (Fc_epsilon_RI) signaling in mast cells, inhibition of Fas/APO-1 apoptotic signaling in B-lineage lymphoid cells, and collagen-stimulated platelet aggregation. See, e.g., C. A. Jeffries, et al., (2003), Journal of Biological Chemistry 278:26258-26264; N. J. Horwood, et al., (2003), The Journal of Experimental Medicine 197: 1603-1611; Iwaki et al. (2005), Journal of Biological Chemistry 280(48):40261-40270; Vassilev et al. (1999), Journal of Biological Chemistry 274(3): 1646-1656, and Quek et al. (1998), Current Biology 8(20): 1137-1140.

Patients with mutations in BTK have a profound block in B cell development, resulting in the almost complete absence of mature B lymphocytes and plasma cells, severely reduced Ig levels and a profound inhibition of humoral response to recall antigens (reviewed in Vihinen et al Frontiers in Bioscience 5: d917-928). Mice deficient in BTK also have a reduced number of peripheral B cells and greatly decreased serum levels of IgM and IgG3. BTK deletion in mice has a profound effect on B cell proliferation induced by anti-IgM, and inhibits immune responses to thymus-independent type II antigens (Ellmeier et al, J Exp Med 192: 1611-1623 (2000)). BTK also plays a crucial role in mast cell activation through the high-affinity IgE receptor (Fc_epsilon_RI). BTK deficient murine mast cells have reduced degranulation and decreased production of proinflammatory cytokines following Fc_epsilon_RI cross-linking (Kawakami et al. Journal of Leukocyte Biology 65: 286-290).

Provided compounds are inhibitors of BTK and are therefore useful for treating one or more disorders associated with activity of BTK. Thus, in some embodiments, the present invention provides a method for treating a BTK-mediated disorder comprising the step of administering to a patient in need thereof a compound of the present invention, or pharmaceutically acceptable composition thereof.

As used herein, the term “BTK-mediated” disorders or conditions as used herein means any disease or other deleterious condition in which BTK, or a mutant thereof, is known or suspected to play a role. Accordingly, another embodiment of the present invention relates to treating or lessening the severity of one or more diseases in which BTK, or a mutant thereof, is known or suspected to play a role. Specifically, the present invention relates to a method of treating or lessening the severity of a disease or condition selected from a proliferative disorder or an autoimmune disorder, wherein said method comprises administering to a patient in need thereof a compound or composition according to the present invention.

In some embodiments, the present invention provides a method for treating or lessening the severity of one or more diseases and conditions associated with BTK. In some embodiments, the disease or condition is an autoimmune disease, e.g., inflammatory bowel disease, arthritis, lupus, rheumatoid arthritis, psoriatic arthritis, osteoarthritis, Still's disease, juvenile arthritis, diabetes, myasthenia gravis, Hashimoto's thyroiditis, Ord's thyroiditis, Graves' disease, Sjogren's syndrome, multiple sclerosis, Guillain-Barre syndrome, acute disseminated encephalomyelitis, Addison's disease, opsoclonus-myoclonus syndrome, ankylosing spondylosis, antiphospholipid antibody syndrome, aplastic anemia, autoimmune hepatitis, celiac disease, Goodpasture's syndrome, idiopathic thrombocytopenic purpura, optic neuritis, scleroderma, primary biliary cirrhosis, Reiter's syndrome, Takayasu's arteritis, temporal arteritis, warm autoimmune hemolytic anemia, Wegener's granulomatosis, psoriasis, alopecia universalis, Behcet's disease, chronic fatigue, dysautonomia, endometriosis, interstitial cystitis, neuromyotonia, scleroderma, or vulvodynia. In some embodiments, the disease or condition is a hyperproliferative disease or immunologically-mediated diseases including rejection of transplanted organs or tissues and Acquired Immunodeficiency Syndrome (AIDS, also known as HIV).

In some embodiments, the present invention provides a method for treating or lessening the severity of one or more diseases and conditions associated with BTK, wherein the disease or condition is selected from heteroimmune conditions or diseases, which include, but are not limited to graft versus host disease, transplantation, transfusion, anaphylaxis, allergies (e.g., allergies to plant pollens, latex, drugs, foods, insect poisons, animal hair, animal dander, dust mites, or cockroach calyx), type I hypersensitivity, allergic conjunctivitis, allergic rhinitis, and atopic dermatitis.

In some embodiments, the present invention provides a method for treating or lessening the severity of one or more diseases and conditions associated with BTK, wherein the disease or condition is selected from an inflammatory disease, e.g., asthma, appendicitis, blepharitis, bronchiolitis, bronchitis, bursitis, cervicitis, cholangitis, cholecystitis, colitis, conjunctivitis, cystitis, dacryoadenitis, dermatitis, dermatomyositis, encephalitis, endocarditis, endometritis, enteritis, enterocolitis, epicondylitis, epididymitis, fasciitis, fibrositis, gastritis, gastroenteritis, hepatitis, hidradenitis suppurativa, laryngitis, mastitis, meningitis, myelitis myocarditis, myositis, nephritis, oophoritis, orchitis, osteitis, otitis, pancreatitis, parotitis, pericarditis, peritonitis, pharyngitis, pleuritis, phlebitis, pneumonitis, pneumonia, proctitis, prostatitis, pyelonephritis, rhinitis, salpingitis, sinusitis, stomatitis, synovitis, tendonitis, tonsillitis, uveitis, vaginitis, vasculitis, or vulvitis.

In some embodiments, the present invention provides a method for treating or lessening the severity of one or more diseases and conditions associated with BTK, wherein the disease or condition is selected from a cancer. In one embodiment, the cancer is a B-cell proliferative disorder, e.g., diffuse large B cell lymphoma, follicular lymphoma, chronic lymphocytic lymphoma, chronic lymphocytic leukemia, small lymphocytic lymphoma, acute lymphocytic leukemia, B-cell prolymphocytic leukemia, lymphoplasmacytic lymphoma/Waldenstrom macroglobulinemia, splenic marginal zone lymphoma, multiple myeloma (also known as plasma cell myeloma), non-Hodgkin's lymphoma, Hodgkin's lymphoma, plasmacytoma, extranodal marginal zone B cell lymphoma, nodal marginal zone B cell lymphoma, mantle cell lymphoma, mediastinal (thymic) large B cell lymphoma, intravascular large B cell lymphoma, primary effusion lymphoma, Burkitt lymphoma/leukemia, or lymphomatoid granulomatosis. In some embodiments, the cancer is breast cancer, prostate cancer, or cancer of the mast cells (e.g., mastocytoma, mast cell leukemia, mast cell sarcoma, systemic mastocytosis). In one embodiment, the cancer is bone cancer. In another embodiment, the cancer is of other primary origin and metastasizes to the bone.

In some embodiments, the present invention provides a method for treating or lessening the severity of one or more diseases or conditions associated with BTK including diseases of the bone and joints including, without limitation, rheumatoid arthritis, seronegative spondyloarthropathies (including ankylosing spondylitis, psoriatic arthritis and Reiter's disease), Behcet's disease, Sjogren's syndrome, systemic sclerosis, osteoporosis, bone cancer, and bone metastasis.

In some embodiments, the present invention provides a method for treating or lessening the severity of one or more diseases and conditions associated with BTK, wherein the disease or condition is selected from a thromboembolic disorder, e.g., myocardial infarct, angina pectoris, reocclusion after angioplasty, restenosis after angioplasty, reocclusion after aortocoronary bypass, restenosis after aortocoronary bypass, stroke, transitory ischemia, a peripheral arterial occlusive disorder, pulmonary embolism, or deep venous thrombosis.

In some embodiments, the present invention provides a method for treating or lessening the severity of one or more diseases and conditions associated with BTK, including infectious and noninfectious inflammatory events and autoimmune and other inflammatory diseases. These autoimmune and inflammatory diseases, disorders, and syndromes include inflammatory pelvic disease, urethritis, skin sunburn, sinusitis, pneumonitis, encephalitis, meningitis, myocarditis, nephritis, osteomyelitis, myositis, hepatitis, gastritis, enteritis, dermatitis, gingivitis, appendicitis, pancreatitis, cholocystitus, agammaglobulinemia, psoriasis, allergy, Crohn's disease, irritable bowel syndrome, ulcerative colitis, Sjogren's disease, tissue graft rejection, hyperacute rejection of transplanted organs, asthma, allergic rhinitis, chronic obstructive pulmonary disease (COPD), autoimmune polyglandular disease (also known as autoimmune polyglandular syndrome), autoimmune alopecia, pernicious anemia, glomerulonephritis, dermatomyositis, multiple sclerosis, scleroderma, vasculitis, autoimmune hemolytic and thrombocytopenic states, Goodpasture's syndrome, atherosclerosis, Addison's disease, Parkinson's disease, Alzheimer's disease, type I diabetes, septic shock, systemic lupus erythematosus (SLE), rheumatoid arthritis, psoriatic arthritis, juvenile arthritis, osteoarthritis, chronic idiopathic thrombocytopenic purpura, Waldenstrom macroglobulinemia, myasthenia gravis, Hashimoto's thyroiditis, atopic dermatitis, degenerative joint disease, vitiligo, autoimmune hypopituitarism, Guillain-Barre syndrome, Behcet's disease, scleraderma, mycosis fungoides, acute inflammatory responses (such as acute respiratory distress syndrome and ischemia/reperfusion injury), and Graves' disease.

In some embodiments, the present invention provides a method for treating or lessening the severity of one or more diseases and conditions associated with BTK, selected from rheumatoid arthritis, multiple sclerosis, B-cell chronic lymphocytic leukemia, acute lymphocytic leukemia, hairy cell leukemia, non-Hodgkin's lymphoma, Hodgkin's lymphoma, multiple myeloma, bone cancer, bone metastasis, osteoporosis, irritable bowel syndrome, Crohn's disease, lupus and renal transplant.

### (d) ITK

Interleukin-2 inducible T-cell kinase (“ITK”) is expressed in T cells, mast cells and natural killer cells. It is activated in T cells upon stimulation of the T cell receptor (TCR), and in mast cells upon activation of the high affinity IgE receptor. Following receptor stimulation in T cells, Lck, a Src tyrosine kinase family member, phosphorylates Y511 in the kinase domain activation loop of ITK (S. D. Heyeck et al., 1997, J. Biol. Chem, 272, 25401-25408). Activated ITK, together with Zap-70 is required for phosphorylation and activation of PLC-gamma (S. C. Bunnell et al., 2000, J. Biol. Chem., 275, 2219-2230). PLC-gamma catalyzes the formation of inositol 1,4,5-triphosphate and diacylglycerol, leading to calcium mobilization and PKC activation, respectively. These events activate numerous downstream pathways and lead ultimately to degranulation (mast cells) and cytokine gene expression (T cells) (Y. Kawakami et al., 1999, J. Leukocyte Biol., 65, 286-290).

The role of ITK in T cell activation has been confirmed in ITK knockout mice. CD4⁺ T cells from ITK knockout mice have a diminished proliferative response in a mixed lymphocyte reaction or upon Con A or anti-CD3 stimulation. (X. C. Liao and D. R. Littman, 1995, Immunity, 3, 757-769). Also, T cells from ITK knockout mice produced little IL-2 upon TCR stimulation resulting in reduced proliferation of these cells. In another study, ITK deficient CD4⁺ T cells produced reduced levels of cytokines including IL-4, IL-5 and IL-13 upon stimulation of the TCR, even after priming with inducing conditions (D. J. Fowell, 1999, Immunity, 11, 399-409).

The role of ITK in PLC-gamma activation and in calcium mobilization was also confirmed in the T cells of these knockout mice, which had severely impaired IP₃ generation and no extracellular calcium influx upon TCR stimulation (K. Liu et al., 1998, J. Exp. Med. 187, 1721-1727). Such studies support a key role for ITK in activation of T cells and mast cells. Thus an inhibitor of ITK would be of therapeutic benefit in diseases mediated by inappropriate activation of these cells.

It has been well established that T cells play an important role in regulating the immune response (Powrie and Coffman, 1993, Immunology Today, 14, 270-274). Indeed, activation of T cells is often the initiating event in immunological disorders. Following activation of the TCR, there is an influx of calcium that is required for T cell activation. Upon activation, T cells produce cytokines, including IL-2, 4, 5, 9, 10, and 13 leading to T cell proliferation, differentiation, and effector function. Clinical studies with inhibitors of IL-2 have shown that interference with T cell activation and proliferation effectively suppresses immune response in vivo (Waldmann, 1993, Immunology Today, 14, 264-270). Accordingly, agents that inhibit T lymphocyte activation and subsequent cytokine production, are therapeutically useful for selectively suppressing the immune response in a patient in need of such immunosuppression.

Mast cells play a critical roll in asthma and allergic disorders by releasing pro-inflammatory mediators and cytokines. Antigen-mediated aggregation of Fc.epsilon.RI, the high-affinity receptor for IgE, results in activation of mast cells (D. B. Corry et al., 1999, Nature, 402, B 18-23). This triggers a series of signaling events resulting in the release of mediators, including histamine, proteases, leukotrienes and cytokines (J. R. Gordon et al., 1990, Immunology Today, 11, 458-464.) These mediators cause increased vascular permeability, mucus production, bronchoconstriction, tissue degradation and inflammation thus playing key roles in the etiology and symptoms of asthma and allergic disorders.

Published data using ITK knockout mice suggests that in the absence of ITK function, increased numbers of memory T cells are generated (A. T. Miller et al., 2002 The Journal of Immunology, 168, 2163-2172). One strategy to improve vaccination methods is to increase the number of memory T cells generated (S. M. Kaech et al., Nature Reviews Immunology, 2, 251-262). In addition, deletion of ITK in mice results in reduced T cell receptor (TCR)-induced proliferation and secretion of the cytokines IL-2, IL-4, IL-5, IL-10 and IFN-y (Schaeffer et al, Science 284; 638-641 (1999)), Fowell et al, Immunity 11, 399-409 (1999), Schaeffer et al, Nature Immunology 2 (12): 1183-1188 (2001))). The immunological symptoms of allergic asthma are attenuated in ITK−/−mice. Lung inflammation, eosinophil infiltration and mucus production are drastically reduced in ITK−/−mice in response to challenge with the allergen OVA (Mueller et al, Journal of Immunology 170: 5056-5063 (2003)). ITK has also been implicated in atopic dermatitis. This gene has been reported to be more highly expressed in peripheral blood T cells from patients with moderate and/or severe atopic dermatitis than in controls or patients with mild atopic dermatitis (Matsumoto et al, International Archives of Allergy and Immunology 129: 327-340 (2002)).

Splenocytes from RLK−/−mice secrete half the IL-2 produced by wild type animals in response to TCR engagement (Schaeffer et al, Science 284: 638-641 (1999)), while combined deletion of ITK and RLK in mice leads to a profound inhibition of TCR-induced responses including proliferation and production of the cytokines IL-2, IL-4, IL-5 and IFN-y (Schaeffer et al, Nature Immunology 2 (12): 1183-1188 (2001), Schaeffer et al, Science 284: 638-641 (1999)). Intracellular signalling following TCR engagement is effected in ITK/RLK deficient T cells; inositol triphosphate production, calcium mobilization, MAP kinase activation, and activation of the transcription factors NFAT and AP-1 are all reduced (Schaeffer et al, Science 284: 638-641 (1999), Schaeffer et al, Nature Immunology 2 (12): 1183-1188 (2001)).

Provided compounds are inhibitors of ITK and are therefore useful for treating one or more disorders associated with activity of ITK. Thus, in some embodiments, the present invention provides a method for treating an ITK-mediated disorder comprising the step of administering to a patient in need thereof a compound of the present invention, or pharmaceutically acceptable composition thereof.

As used herein, the term “ITK-mediated” disorders or conditions as used herein means any disease or other deleterious condition in which ITK, or a mutant thereof, is known or suspected to play a role. Accordingly, another embodiment of the present invention relates to treating or lessening the severity of one or more diseases in which ITK, or a mutant thereof, is known or suspected to play a role. Specifically, the present invention relates to a method of treating or lessening the severity of a disease or condition selected from a mast cell-mediated condition, a basophil-mediated disorder, an immune or allergic disorder, wherein said method comprises administering to a patient in need thereof a compound or composition according to the present invention.

In some embodiments, the present invention provides a method for treating or lessening the severity of one or more diseases and conditions associated with ITK, wherein the disease or condition is an immune disorder, including inflammatory diseases, autoimmune diseases, organ and bone marrow transplant rejection and other disorders associated with T cell-mediated immune response or mast cell-mediated immune response.

In certain embodiments, the present invention provides a method for treating or lessening the severity of one or more diseases and conditions associated with ITK, wherein the disease or condition is acute or chronic inflammation, an allergy, contact dermatitis, psoriasis, rheumatoid arthritis, multiple sclerosis, type 1 diabetes, inflammatory bowel disease, Guillain-Barre syndrome, Crohn's disease, ulcerative colitis, cancer, graft versus host disease (and other forms of organ or bone marrow transplant rejection) or lupus erythematosus.

In certain embodiments, the present invention provides a method for treating or lessening the severity of one or more diseases and conditions associated with ITK, wherein the disease or condition is a mast cell driven conditions, a basophil-mediated disorder, reversible obstructive airway disease, asthma, rhinitis, chronic obstructive pulmonary disease (COPD), peripheral T-cell lymphomas or HIV [also known as Acquired Immunodeficiency Syndrome (AIDS)]. Such conditions include those described in Readinger, et al., PNAS 105: 6684-6689 (2008).

### (e) JAK Family

The Janus kinases (JAK) are a family of tyrosine kinases consisting of JAK1, JAK2, JAK3 and TYK2. The JAKs play a critical role in cytokine signaling. The down-stream substrates of the JAK family of kinases include the signal transducer and activator of transcription (STAT) proteins. JAK/STAT signaling has been implicated in the mediation of many abnormal immune responses such as allergies, asthma, autoimmune diseases such as transplant rejection, rheumatoid arthritis, amyotrophic lateral sclerosis and multiple sclerosis as well as in solid and hematologic malignancies such as leukemias and lymphomas. The pharmaceutical intervention in the JAK/STAT pathway has been reviewed [Frank, Mol. Med. 5: 432-456 (1999) & Seidel, et al, Oncogene 19: 2645-2656 (2000)].

JAK1, JAK2, and TYK2 are ubiquitously expressed, while JAK3 is predominantly expressed in hematopoietic cells. JAK3 binds exclusively to the common cytokine receptor gamma chain (yc) and is activated by IL-2, IL-4, IL-7, IL-9, and IL-15.

The proliferation and survival of murine mast cells induced by IL-4 and IL-9 have, in fact, been shown to be dependent on JAK3- and yc-signaling [Suzuki et al, Blood 96: 2172-2180 (2000)].

Cross-linking of the high-affinity immunoglobulin (Ig) E receptors of sensitized mast cells leads to a release of proinflammatory mediators, including a number of vasoactive cytokines resulting in acute allergic, or immediate (type I) hypersensitivity reactions [Gordon et al, Nature 346: 274-276 (1990) & Galli, N. Engl. J. Med., 328: 257-265 (1993)]. A crucial role for JAK3 in IgE receptor-mediated mast cell responses in vitro and in vivo has been established [Malaviya, et al, Biochem. Biophys. Res. Commun. 257: 807-813 (1999)]. In addition, the prevention of type I hypersensitivity reactions, including anaphylaxis, mediated by mast cell-activation through inhibition of JAK3 has also been reported [Malaviya et al, J. Biol. Chem. 274: 27028-27038 (1999)]. Targeting mast cells with JAK3 inhibitors modulated mast cell degranulation in vitro and prevented IgE receptor/antigen-mediated anaphylactic reactions in vivo.

A recent study described the successful targeting of JAK3 for immune suppression and allograft acceptance. The study demonstrated a dose-dependent survival of buffalo heart allograft in Wistar Furth recipients upon administration of inhibitors of JAK3 indicating the possibility of regulating unwanted immune responses in graft versus host disease [Kirken, Transpl. Proc. 33: 3268-3270 (2001)].

IL-4-mediated STAT-phosphorylation has been implicated as the mechanism involved in early and late stages of rheumatoid arthritis (RA). Up-regulation of proinflammatory cytokines in RA synovium and synovial fluid is a characteristic of the disease. It has been demostrated that IL-4-mediated activation of IL-4/STAT pathway is mediated through the Janus kinases (JAK 1 & 3) and that IL-4-associated JAK kinases are expressed in the RA synovium [Muller-Ladner, et al, J. Immunol. 164: 3894-3901 (2000)].

Familial amyotrophic lateral sclerosis (FALS) is a fatal neurodegenerative disorder affecting about 10% of ALS patients. The survival rates of FALS mice were increased upon treatment with a JAK3 specific inhibitor. This confirmed that JAK3 plays a role in FALS [Trieu, et al, Biochem. Biophys. Res. Commun. 267: 22-25 (2000)].

Signal transducer and activator of transcription (STAT) proteins are activated by, among others, the JAK family kinases. Results form a recent study suggested the possibility of intervention in the JAK/STAT signaling pathway by targeting JAK family kinases with specific inhibitors for the treatment of leukemia [Sudbeck, et al., Clin. Cancer Res. 5: 1569-1582 (1999)]. JAK3 specific compounds were shown to inhibit the clonogenic growth of JAK3-expressing cell lines DAUDI, RAMOS, LC1; 19, NALM-6, MOLT-3 and HL-60. Inhibition of JAK3 and TYK 2 abrogated tyrosine phosphorylation of STAT3, and inhibited cell growth of mycosis fungoides, a form of cutaneous T cell lymphoma.

According to another embodiment, the invention provides a method for treating or lessening the severity of a JAK3-mediated disease or condition in a patient comprising the step of administering to said patient a composition according to the present invention.

The term “JAK3-mediated disease”, as used herein means any disease or other deleterious condition in which a JAK3 kinase is known or suspected to play a role. Accordingly, another embodiment of the present invention relates to treating or lessening the severity of one or more diseases in which JAK3 is known or suspected to play a role. Specifically, the present invention relates to a method of treating or lessening the severity of a disease or condition selected from immune responses such as allergic or type I hypersensitivity reactions, asthma, autoimmune diseases such as transplant rejection, graft versus host disease, rheumatoid arthritis, amyotrophic lateral sclerosis, and multiple sclerosis, neurodegenerative disorders such as familial amyotrophic lateral sclerosis (FALS), as well as in solid and hematologic malignancies such as leukemias and lymphomas, wherein said method comprises administering to a patient in need thereof a composition according to the present invention.

The compounds and compositions, according to the method of the present invention, may be administered using any amount and any route of administration effective for treating or lessening the severity of cancer, an autoimmune disorder, a neurodegenerative or neurological disorder, schizophrenia, a bone-related disorder, liver disease, or a cardiac disorder. The exact amount required will vary from subject to subject, depending on the species, age, and general condition of the subject, the severity of the infection, the particular agent, its mode of administration, and the like. Compounds of the invention are preferably formulated in dosage unit form for ease of administration and uniformity of dosage. The expression “dosage unit form” as used herein refers to a physically discrete unit of agent appropriate for the patient to be treated. It will be understood, however, that the total daily usage of the compounds and compositions of the present invention will be decided by the attending physician within the scope of sound medical judgment. The specific effective dose level for any particular patient or organism will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed, and like factors well known in the medical arts. The term “patient”, as used herein, means an animal, preferably a mammal, and most preferably a human.

Pharmaceutically acceptable compositions of this invention can be administered to humans and other animals orally, rectally, parenterally, intracisternally, intravaginally, intraperitoneally, topically (as by powders, ointments, or drops), bucally, as an oral or nasal spray, or the like, depending on the severity of the infection being treated. In certain embodiments, the compounds of the invention may be administered orally or parenterally at dosage levels of about 0.01 mg/kg to about 50 mg/kg and preferably from about 1 mg/kg to about 25 mg/kg, of subject body weight per day, one or more times a day, to obtain the desired therapeutic effect.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

Injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a compound of the present invention, it is often desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsule matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

Compositions for rectal or vaginal administration are preferably suppositories which can be prepared by mixing the compounds of this invention with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polethylene glycols and the like.

The active compounds can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of a compound of this invention include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, and eye drops are also contemplated as being within the scope of this invention. Additionally, the present invention contemplates the use of transdermal patches, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

According to one embodiment, the invention relates to a method of inhibiting protein kinase activity in a biological sample comprising the step of contacting said biological sample with a compound of this invention, or a composition comprising said compound.

According to another embodiment, the invention relates to a method of inhibiting ErbB1, ErbB2, ErbB3, ErbB4, a TEC-kinase (e.g., TEC, BTK, ITK, RLK or BMX), and/or JAK3, or a mutant thereof, activity in a biological sample comprising the step of contacting said biological sample with a compound of this invention, or a composition comprising said compound. In certain embodiments, the invention relates to a method of irreversibly inhibiting ErbB1, ErbB2, ErbB3, ErbB4, a TEC-kinase (e.g., TEC, BTK, ITK, RLK or BMX), and/or JAK3, or a mutant thereof, activity in a biological sample comprising the step of contacting said biological sample with a compound of this invention, or a composition comprising said compound.

The term “biological sample”, as used herein, includes, without limitation, cell cultures or extracts thereof; biopsied material obtained from a mammal or extracts thereof; and blood, saliva, urine, feces, semen, tears, or other body fluids or extracts thereof.

Inhibition of protein kinase, or a protein kinase selected from ErbB1, ErbB2, ErbB3, ErbB4, a TEC-kinase (i.e TEC, BTK, ITK, RLK or BMX), and/or JAK3, or a mutant thereof, activity in a biological sample is useful for a variety of purposes that are known to one of skill in the art. Examples of such purposes include, but are not limited to, blood transfusion, organ transplantation, biological specimen storage, and biological assays.

Another embodiment of the present invention relates to a method of inhibiting protein kinase activity in a patient comprising the step of administering to said patient a compound of the present invention, or a composition comprising said compound.

According to another embodiment, the invention relates to a method of inhibiting one or more of ErbB1, ErbB2, ErbB3, ErbB4, a TEC-kinase (e.g., TEC, BTK, ITK, RLK or BMX), and/or JAK3, or a mutant thereof, activity in a patient comprising the step of administering to said patient a compound of the present invention, or a composition comprising said compound. According to certain embodiments, the invention relates to a method of irreversibly inhibiting one or more of ErbB1, ErbB2, ErbB3, ErbB4, a TEC-kinase (e.g., TEC, BTK, ITK, RLK or BMX), and/or JAK3, or a mutant thereof, activity in a patient comprising the step of administering to said patient a compound of the present invention, or a composition comprising said compound. In other embodiments, the present invention provides a method for treating a disorder mediated by one or more of ErbB1, ErbB2, ErbB3, ErbB4, a TEC-kinase (e.g., TEC, BTK, ITK, RLK or BMX), and/or JAK3, or a mutant thereof, in a patient in need thereof, comprising the step of administering to said patient a compound according to the present invention or pharmaceutically acceptable composition thereof. Such disorders are described in detail herein.

Depending upon the particular condition, or disease, to be treated, additional therapeutic agents, which are normally administered to treat that condition, may also be present in the compositions of this invention. As used herein, additional therapeutic agents that are normally administered to treat a particular disease, or condition, are known as “appropriate for the disease, or condition, being treated.”

For example, compounds of the present invention, or a pharmaceutically acceptable composition thereof, are administered in combination with chemotherapeutic agents to treat proliferative diseases and cancer. Examples of known chemotherapeutic agents include, but are not limited to, Adriamycin, dexamethasone, vincristine, cyclophosphamide, fluorouracil, topotecan, taxol, interferons, platinum derivatives, taxane (e.g., paclitaxel), vinca alkaloids (e.g., vinblastine), anthracyclines (e.g., doxorubicin), epipodophyllotoxins (e.g., etoposide), cisplatin, an mTOR inhibitor (e.g., a rapamycin), methotrexate, actinomycin D, dolastatin 10, colchicine, emetine, trimetrexate, metoprine, cyclosporine, daunorubicin, teniposide, amphotericin, alkylating agents (e.g., chlorambucil), 5-fluorouracil, campthothecin, cisplatin, metronidazole, and Gleevec™, among others. In other embodiments, a compound of the present invention is administered in combination with a biologic agent, such as Avastin or VECTIBIX.

In certain embodiments, compounds of the present invention, or a pharmaceutically acceptable composition thereof, are administered in combination with an antiproliferative or chemotherapeutic agent selected from any one or more of abarelix, aldesleukin, alemtuzumab, alitretinoin, allopurinol, altretamine, amifostine, anastrozole, arsenic trioxide, asparaginase, azacitidine, BCG Live, bevacuzimab, fluorouracil, bexarotene, bleomycin, bortezomib, busulfan, calusterone, capecitabine, camptothecin, carboplatin, carmustine, celecoxib, cetuximab, chlorambucil, cladribine, clofarabine, cyclophosphamide, cytarabine, dactinomycin, darbepoetin alfa, daunorubicin, denileukin, dexrazoxane, docetaxel, doxorubicin (neutral), doxorubicin hydrochloride, dromostanolone propionate, epirubicin, epoetin alfa, erlotinib, estramustine, etoposide phosphate, etoposide, exemestane, filgrastim, floxuridine fludarabine, fulvestrant, gefitinib, gemcitabine, gemtuzumab, goserelin acetate, histrelin acetate, hydroxyurea, ibritumomab, idarubicin, ifosfamide, imatinib mesylate, interferon alfa-2a, interferon alfa-2b, irinotecan, lenalidomide, letrozole, leucovorin, leuprolide acetate, levamisole, lomustine, megestrol acetate, melphalan, mercaptopurine, 6-MP, mesna, methotrexate, methoxsalen, mitomycin C, mitotane, mitoxantrone, nandrolone, nelarabine, nofetumomab, oprelvekin, oxaliplatin, paclitaxel, palifermin, pamidronate, pegademase, pegaspargase, pegfilgrastim, pemetrexed disodium, pentostatin, pipobroman, plicamycin, porfimer sodium, procarbazine, quinacrine, rasburicase, rituximab, sargramostim, sorafenib, streptozocin, sunitinib maleate, talc, tamoxifen, temozolomide, teniposide, VM-26, testolactone, thioguanine, 6-TG, thiotepa, topotecan, toremifene, tositumomab, trastuzumab, tretinoin, ATRA, uracil mustard, valrubicin, vinblastine, vincristine, vinorelbine, zoledronate, or zoledronic acid.

Other examples of agents the inhibitors of this invention may also be combined with include, without limitation: treatments for Alzheimer's Disease such as donepezil hydrochloride (Aricept®) and rivastigmine (Exelon®); treatments for Parkinson's Disease such as L-DOPA/carbidopa, entacapone, ropinrole, pramipexole, bromocriptine, pergolide, trihexephendyl, and amantadine; agents for treating Multiple Sclerosis (MS) such as beta interferon (e.g., Avonex® and Rebif®), glatiramer acetate (Copaxone®), and mitoxantrone; treatments for asthma such as albuterol and montelukast (Singulair®); agents for treating schizophrenia such as zyprexa, risperdal, seroquel, and haloperidol; anti-inflammatory agents such as corticosteroids, TNF blockers, IL-1 RA, azathioprine, cyclophosphamide, and sulfasalazine; immunomodulatory and immunosuppressive agents such as cyclosporin, tacrolimus, rapamycin, mycophenolate mofetil, interferons, corticosteroids, cyclophophamide, azathioprine, and sulfasalazine; neurotrophic factors such as acetylcholinesterase inhibitors, MAO inhibitors, interferons, anti-convulsants, ion channel blockers, riluzole, and anti-Parkinsonian agents; agents for treating cardiovascular disease such as beta-blockers, ACE inhibitors, diuretics, nitrates, calcium channel blockers, and statins; agents for treating liver disease such as corticosteroids, cholestyramine, interferons, and anti-viral agents; agents for treating blood disorders such as corticosteroids, anti-leukemic agents, and growth factors; and agents for treating immunodeficiency disorders such as gamma globulin.

In certain embodiments, compounds of the present invention, or a pharmaceutically acceptable composition thereof, are administered in combination with a monoclonal antibody or an siRNA therapeutic.

Those additional agents may be administered separately from an inventive compound-containing composition, as part of a multiple dosage regimen. Alternatively, those agents may be part of a single dosage form, mixed together with a compound of this invention in a single composition. If administered as part of a multiple dosage regime, the two active agents may be submitted simultaneously, sequentially or within a period of time from one another normally within five hours from one another.

As used herein, the term “combination,” “combined,” and related terms refers to the simultaneous or sequential administration of therapeutic agents in accordance with this invention. For example, a compound of the present invention may be administered with another therapeutic agent simultaneously or sequentially in separate unit dosage forms or together in a single unit dosage form. Accordingly, the present invention provides a single unit dosage form comprising a provided compound, an additional therapeutic agent, and a pharmaceutically acceptable carrier, adjuvant, or vehicle.

The amount of both, an inventive compound and additional therapeutic agent (in those compositions which comprise an additional therapeutic agent as described above)) that may be combined with the carrier materials to produce a single dosage form will vary depending upon the host treated and the particular mode of administration. Preferably, compositions of this invention should be formulated so that a dosage of between 0.01-100 mg/kg body weight/day of an inventive can be administered.

In those compositions which comprise an additional therapeutic agent, that additional therapeutic agent and the compound of this invention may act synergistically. Therefore, the amount of additional therapeutic agent in such compositions will be less than that required in a monotherapy utilizing only that therapeutic agent. In such compositions a dosage of between 0.01-1,000 rig/kg body weight/day of the additional therapeutic agent can be administered.

The amount of additional therapeutic agent present in the compositions of this invention will be no more than the amount that would normally be administered in a composition comprising that therapeutic agent as the only active agent. Preferably the amount of additional therapeutic agent in the presently disclosed compositions will range from about 50% to 100% of the amount normally present in a composition comprising that agent as the only therapeutically active agent.

The compounds of this invention, or pharmaceutical compositions thereof, may also be incorporated into compositions for coating an implantable medical device, such as prostheses, artificial valves, vascular grafts, stents and catheters. Vascular stents, for example, have been used to overcome restenosis (re-narrowing of the vessel wall after injury). However, patients using stents or other implantable devices risk clot formation or platelet activation. These unwanted effects may be prevented or mitigated by pre-coating the device with a pharmaceutically acceptable composition comprising a kinase inhibitor. Implantable devices coated with a compound of this invention are another embodiment of the present invention.

### EXEMPLIFICATION

As depicted in the Examples below, in certain exemplary embodiments, compounds are prepared according to the following general procedures. It will be appreciated that, although the general methods depict the synthesis of certain compounds of the present invention, the following general methods, and other methods known to one of ordinary skill in the art, can be applied to all compounds and subclasses and species of each of these compounds, as described herein.

### Example 1

### Preparation of Deuterated Aniline Intermediate 1

The deuterated aniline intermediate (1) was prepared according to the below scheme:

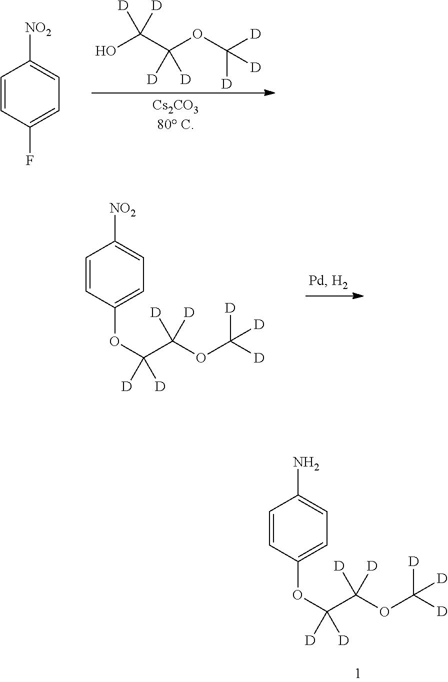 [SMILES: O=[N+]([O-])c1ccc(F)cc1 ; [2H]C([2H])([2H])OC([2H])([2H])C([2H])([2H])O ; [2H]C([2H])([2H])OC([2H])([2H])C([2H])([2H])Oc1ccc([N+](=O)[O-])cc1 ; [2H]C([2H])([2H])OC([2H])([2H])C([2H])([2H])Oc1ccc(N)cc1]

### Example 2

### Preparation of Deuterated Aniline Intermediate 2

The deuterated aniline intermediate (2) is prepared according to the below scheme:

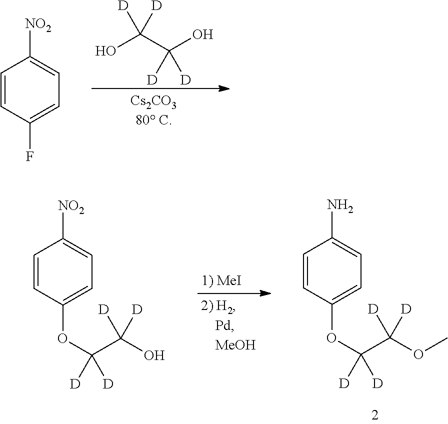 [SMILES: O=[N+]([O-])c1ccc(F)cc1 ; [2H]C([2H])(O)C([2H])([2H])O ; [2H]C([2H])(O)C([2H])([2H])Oc1ccc([N+](=O)[O-])cc1 ; [2H]C([2H])(OC)C([2H])([2H])Oc1ccc(N)cc1]

### Example 3

### Preparation of Deuterated Aniline Intermediate 3

The deuterated aniline intermediate (3) is prepared according to the below scheme:

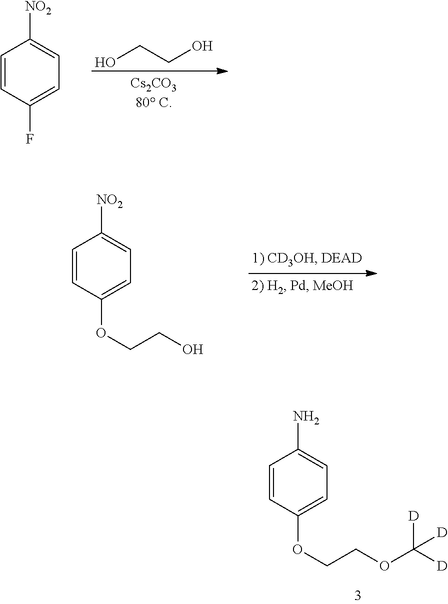

### Example 4

### Preparation of Compound 11-7

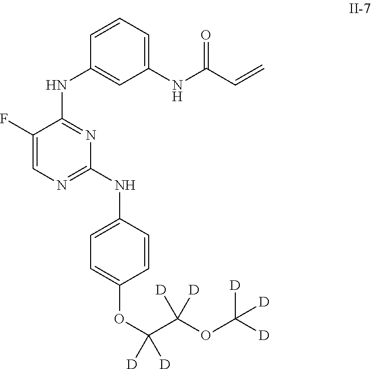 [SMILES: [2H]C([2H])([2H])OC([2H])([2H])C([2H])([2H])Oc1ccc(Nc2ncc(F)c(Nc3cccc(NC(=O)C=C)c3)n2)cc1]

The title compound was prepared according to the schemes, steps and intermediates described below.

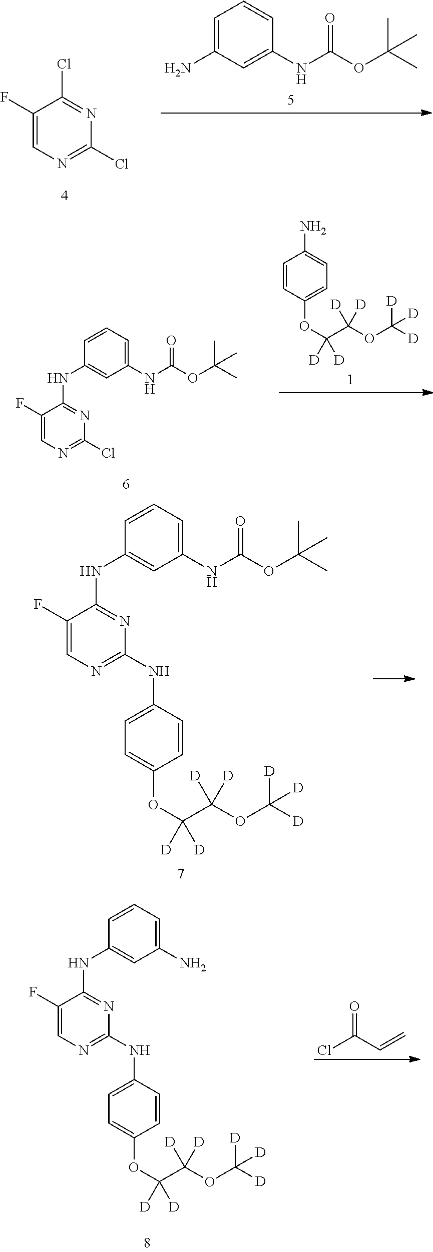

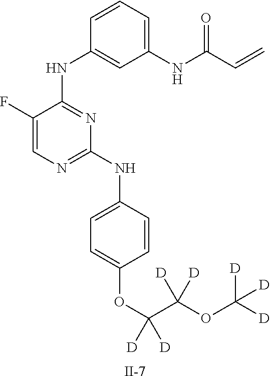

2,4-Dichloro-5-fluoropyrimidine (4), tert-butyl(3-aminophenyl)carbamate (5) and Hunig's base are dissolved in THF. The reaction mixture is heated at reflux overnight. After cooling, the reaction mixture is partitioned between water/brine, agitated and the layers are separated. The organic phase is dried over sodium sulfate, and the solvent is removed via rotary evaporation to afford tert-butyl (3-((2-chloro-5-fluoropyrimidin-4-yl)amino)phenyl)carbamate (6).

tert-Butyl (3-((2-chloro-5-fluoropyrimidin-4-yl)amino)phenyl)carbamate (6) and deuterated aniline 1 are suspended in tert-amyl alcohol and acetic acid. The reaction is heated to reflux for 4 h. After cooling, solvent is removed via rotary evaporation. The mixture is partitioned between water/brine and THF, agitated, and the layers are separated and dried organic phase over sodium sulfate. The solvent is removed via rotary evaporation to afford intermediate 7.

To a solution of intermediate 7 in DCM is added TFA. The reaction is stirred at room temperature and monitored for completion. Once the reaction is complete, the solvent is removed via rotary evaporation and partitioned oil with cold (0° C.) saturated sodium bicarbonate and EtOAc. The mixture is agitated and the layers are separated. The organic phase is dried over sodium sulfate and the solvent removed via rotary evaporation to afford intermediate 8.

A solution of intermediate 8 in THF is cooled in a water/ice-MeOH bath (−10° C.). To this solution is added acryloyl chloride. The reaction is stirred for 10 min, then Hunig's base is added and the reaction is stirred for an additional 10 min. The reaction mixture is partitioned between water/brine, agitated and the layers are separated. The organic phase is dried over sodium sulphate and the solvent is removed via rotary evaporation to afford Compound 11-7.

### Example 5

### Alternative Preparation of Compound 11-7

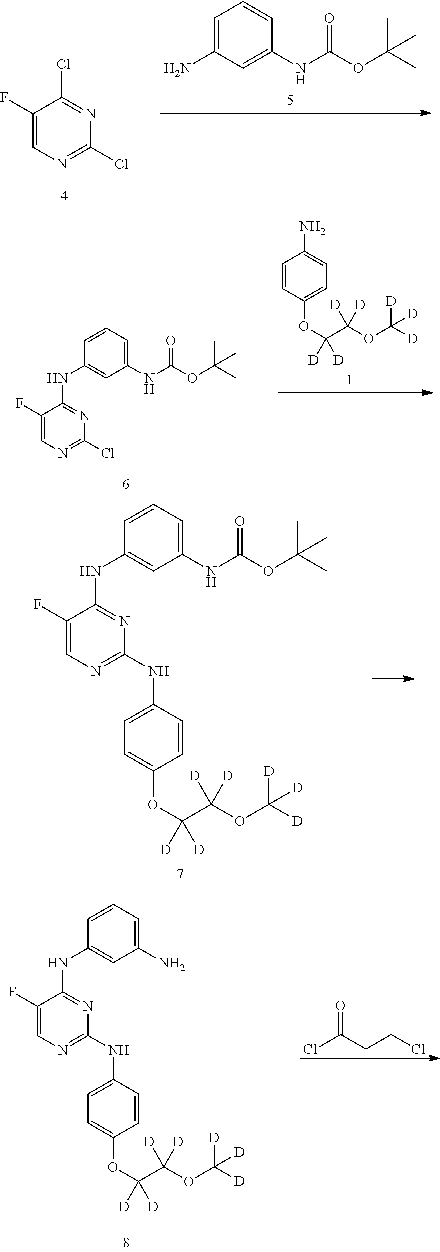

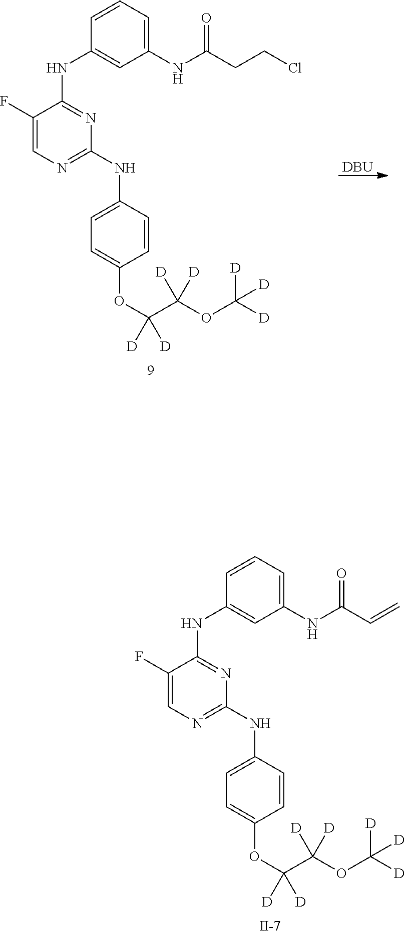

Intermediate 8 was prepared as described above in Example 4. Intermediate 8 was treated with 3-chloropropanoyl chloride to afford intermediate 9, which was isolated and purified. Treatment of intermediate 9 with 1,8-diazabicyclo[5.4.0]undec-7-ene (DBU) afforded Compound 11-7.

### Example 6

### Preparation of Compound 11-4

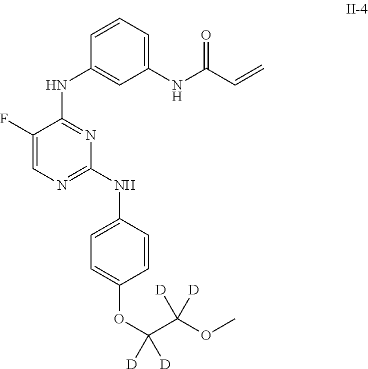 [SMILES: [2H]C([2H])(OC)C([2H])([2H])Oc1ccc(Nc2ncc(F)c(Nc3cccc(NC(=O)C=C)c3)n2)cc1]

Compound II-4 is prepared according to the schemes, steps and intermediates described in Example 4 using intermediate 2 in place of intermediate 1.

### Example 7

### Preparation of Compound 11-3

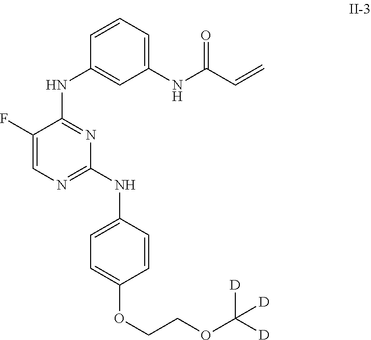 [SMILES: [2H]C([2H])([2H])OCCOc1ccc(Nc2ncc(F)c(Nc3cccc(NC(=O)C=C)c3)n2)cc1]

Compound II-3 is prepared according to the schemes, steps and intermediates described in Example 4 using intermediate 3 in place of intermediate 1.

### Example 8

### Omnia Assay Protocol for Potency Assessment Against BTK and EGFR

The protocol for EGFR-WT potency assessment of the compounds against EGFR is described below.

The mechanics of the assay platform are best described by the vendor (Invitrogen, Carlsbad, Calif.).

10× stocks of EGFR-WT (PV3872) from Invitrogen, 1.13×ATP (Invitrogen catalog number AS001A) and appropriate Tyr-Sox conjugated peptide substrates (Invitrogen catalog number KCZ1001) were prepared in 1× kinase reaction buffer consisting of 20 mM Tris, pH 7.5, 5 mM MgCl₂, 1 mM EGTA, 5 mM β-glycerophosphate, 5% glycerol (10× stock, Invitrogen catalog number KB002A) and 0.2 mM DTT (Invitrogen catalog number DS001A). 5 μL of each enzyme were pre-incubated in a Corning (#3574) 384-well, white, non-binding surface microtiter plate (Corning, N.Y.) for 30 min. at 27° C. with a 0.5 μL volume of 50% DMSO and serially diluted compounds prepared in 50% DMSO. Kinase reactions were started with the addition of 45 μL of the ATP/Tyr-Sox peptide substrate mix and monitored every 30-90 seconds for 60 minutes at λₑₓ360/λₑₘ485 in a Synergy⁴ plate reader from BioTek (Winooski, Vt.). At the conclusion of each assay, progress curves from each well were examined for linear reaction kinetics and fit statistics (R², 95% confidence interval, absolute sum of squares). Initial velocity (0 minutes to ˜30 minutes) from each reaction was determined from the slope of a plot of relative fluorescence units vs time (minutes) and then plotted against inhibitor concentration to estimate IC₅₀ from log[Inhibitor] vs Response, Variable Slope model in GraphPad Prism from GraphPad Software (San Diego, Calif.).

BTK potency was assessed using the same protocol as that described above for EGFR-WT with the following BTK-optimized reagent conditions: [BTK]=5 nM, [ATP]=40 mM, [Y5-Sox]=10 mM (ATP KMapp ˜36 mM).

Table 21 shows the activity of selected compounds of this invention in the EGFR and BTK inhibition assays. The compound numbers correspond to the compound numbers in Table 5. Compounds having an activity designated as “A” provided an IC₅₀≦10 nM; compounds having an activity designated as “B” provided an IC₅₀ 10-100 nM; compounds having an activity designated as “C” provided an IC₅₀ of 100-1000 nM; compounds having an activity designated as “D” provided an IC₅₀ of 1000-10,000 nM; and compounds having an activity designated as “E” provided an IC₅₀≧10,000 nM.

TABLE 21
EGFR and BTK Inhibition Data

| Compound No. | BTK Inhibition | EGFR Inhibition |
|---|---|---|
| II-7 | A | B |

While a number of embodiments of this invention are described herein, it is apparent that the basic examples may be altered to provide other embodiments that utilize the compounds and methods of this invention. Therefore, it will be appreciated that the scope of this invention is to be defined by the appended claims rather than by the specific embodiments that have been represented by way of example.

We claim:

1. A compound of Formula I: 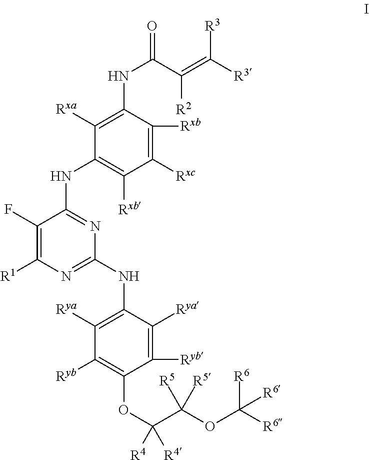
or a pharmaceutically acceptable salt thereof, wherein:
each R¹, R², R³, R^{3′}, R⁴, R^{4′}, R⁵, R^{5′}, R⁶, R^{6′}, R^{6″}, R^{xa}, R^{xb}, R^{xb′}, R^{xc}, R^{ya}, R^{ya′}, R^{yb} and R^{yb′} is independently selected from hydrogen or deuterium;
provided that at least one of R¹, R², R³, R^{3′}, R⁴, R^{4′}, R⁵, R^{5′}, R⁶, R^{6′}, R^{6″}, R^{xa}, R^{xb}, R^{xb′}, R^{xc}, R^{ya}, R^{ya′}, R^{yb} and R^{yb′} is deuterium.

2. The compound according to claim 1, wherein each of R², R³ and R^{3′} is hydrogen.

3. The compound according to claim 1 or claim 2, wherein R⁴ and R^{4′} are the same.

4. The compound according to any of claims 1-3, wherein R⁵ and R^{5′} are the same.

5. The compound according to any of claims 1-4, wherein R⁶, R^{6′} and R^{6″} are the same.

6. The compound according to any of claims 1-5, wherein each of R⁴ and R^{4′} is deuterium.

7. The compound according to any of claims 1-6, wherein each of R⁵ and R^{5′} is deuterium.

8. The compound according to any of claims 1-7, wherein each of R⁶, R^{6′} and R^{6″} is deuterium.

9. The compound according to any of claims 1-5, wherein each of R⁴ and R^{4′} is hydrogen.

10. The compound according to any of claims 1-6, wherein each of R⁵ and R^{5′} is hydrogen.

11. The compound according to any of claims 1-7, wherein each of R⁶, R^{6′} and R^{6″} is hydrogen.

12. The compound according to any of claims 1-11, wherein R¹ is deuterium.

13. The compound according to any of claims 1-11, wherein R¹ is hydrogen.

14. The compound according to any of claims 1-13, wherein R^{xa} is deuterium.

15. The compound according to any of claims 1-14, wherein each of R^{xb} and R^{xb′} is deuterium.

16. The compound according to any of claims 1-15, wherein R^{xc} is deuterium.

17. The compound according to any of claims 1-13, wherein R^{xa} is hydrogen.

18. The compound according to any of claims 1-14, wherein each of R^{xb} and R^{xb′} is hydrogen.

19. The compound according to any of claims 1-15, wherein R^{xc} is hydrogen.

20. The compound according to any of claims 1-19, wherein each of R^{ya} and R^{ya′} is deuterium.

21. The compound according to any of claims 1-20, wherein each of R^{yb} and R^{yb′} is deuterium.

22. The compound according to any of claims 1-19, wherein each of R^{ya} and R^{ya′} is hydrogen.

23. The compound according to any of claims 1-20, wherein each of R^{yb} and R^{yb′} is hydrogen.

24. The compound according to claim 2, wherein each of R¹, R^{xa}, R^{xb}, R^{xb′}, R^{xc}, R^{ya}, R^{ya′}, R^{yb} and R^{yb′} is hydrogen, and the compound is selected from any of the compounds in the following table:

| Compound # | R⁴/R^{4′} | R⁵/R^{5′} | R⁶/R^{6′}/R^{6″} |
|---|---|---|---|
| II-1 | D | H | H |
| II-2 | H | D | H |
| II-3 | H | H | D |
| II-4 | D | D | H |
| II-5 | H | D | D |
| II-6 | D | H | D |
| II-7 | D | D | D |

25. The compound according to claim 2, wherein R¹ is deuterium, each of R^{xa}, R^{xb}, R^{xb′}, R^{xc}, R^{ya}, R^{ya′}, R^{yb} and R^{yb′} is hydrogen, and the compound is selected from any of the compounds in the following table:

| Compound # | R⁴/R^{4′} | R⁵/R^{5′} | R⁶/R^{6′}/R^{6″} |
|---|---|---|---|
| II-8 | H | H | H |
| II-9 | D | H | H |
| II-10 | H | D | H |
| II-11 | H | H | D |
| II-12 | D | D | H |
| II-13 | D | H | D |
| II-14 | H | D | D |
| II-15 | D | D | D |

26. The compound according to claim 2, wherein each of R¹, R⁴, R^{4′}, R⁵, R^{5′}, R⁶, R^{6′}, R^{6″}, R^{ya}, R^{ya′}, R^{yb} and R^{yb′} is hydrogen, and the compound is selected from any of the compounds in the following table:

| Compound # | R^{xa} | R^{xb}/R^{xb′} | R^{xc} |
|---|---|---|---|
| III-1 | D | H | H |
| III-2 | H | D | H |
| III-3 | H | H | D |
| III-4 | D | D | H |
| III-5 | H | D | D |
| III-6 | D | H | D |
| III-7 | D | D | D |

27. The compound according to claim 2, wherein each of R¹, R⁴, R^{4′}, R⁵, R^{5′}, R⁶, R^{6′}, R^{6″}, R^{xa}, R^{xb}, R^{xb′} and R^{xc} is hydrogen, and the compound is selected from any of the compounds in the following table:

| Compound # | R^{ya}/R^{ya′} | R^{yb}/R^{yb′} |
|---|---|---|
| IV-1 | D | H |
| IV-2 | H | D |
| IV-3 | D | D |

28. The compound according to claim 2, wherein each of R⁴, R^{4′}, R⁵, R^{5′}, R⁶, R^{6′}, R^{6″}, R^{ya}, R^{ya′}, R^{yb} and R^{yb′} is hydrogen, and the compound is selected from any of the compounds in the following table:

| Compound # | R¹ | R^{xa} | R^{xb}/R^{xb′} | R^{xc} |
|---|---|---|---|---|
| V-1 | D | D | H | H |
| V-2 | D | H | D | H |
| V-3 | D | H | H | D |
| V-4 | D | D | D | H |
| V-5 | D | D | H | D |
| V-6 | D | H | D | D |
| V-7 | D | D | D | D |

29. The compound according to claim 2, wherein each of R¹, R^{ya}, R^{ya′}, R^{yb} and R^{yb′} is hydrogen, and the compound is selected from any of the compounds in the following table:

| Compound # | R⁴/R^{4′} | R⁵/R^{5′} | R⁶/R^{6′}/R^{6″} | R^{xa} | R^{xb}/R^{xb′} | R^{xc} |
|---|---|---|---|---|---|---|
| V-8 | D | H | H | D | H | H |
| V-9 | D | H | H | H | D | H |
| V-10 | D | H | H | H | H | D |
| V-11 | D | H | H | D | D | H |
| V-12 | D | H | H | D | H | D |
| V-13 | D | H | H | H | D | D |
| V-14 | D | H | H | D | D | D |
| V-15 | H | D | H | D | H | H |
| V-16 | H | D | H | H | D | H |
| V-17 | H | D | H | H | H | D |
| V-18 | H | D | H | D | D | H |
| V-19 | H | D | H | D | H | D |
| V-20 | H | D | H | H | D | D |
| V-21 | H | D | H | D | D | D |
| V-22 | H | H | D | D | H | H |
| V-23 | H | H | D | H | D | H |
| V-24 | H | H | D | H | H | D |
| V-25 | H | H | D | D | D | H |
| V-26 | H | H | D | D | H | D |
| V-27 | H | H | D | H | D | D |
| V-28 | H | H | D | D | D | D |
| V-29 | D | D | H | D | H | H |
| V-30 | D | D | H | H | D | H |
| V-31 | D | D | H | H | H | D |
| V-32 | D | D | H | D | D | H |
| V-33 | D | D | H | H | D | D |
| V-34 | D | D | H | D | H | D |
| V-35 | D | D | H | D | D | D |
| V-36 | H | D | D | D | H | H |
| V-37 | H | D | D | H | D | H |
| V-38 | H | D | D | H | H | D |
| V-39 | H | D | D | D | D | H |
| V-40 | H | D | D | D | H | D |
| V-41 | H | D | D | H | D | D |
| V-42 | H | D | D | D | D | D |
| V-43 | D | H | D | D | H | H |
| V-44 | D | H | D | H | D | H |
| V-45 | D | H | D | H | H | D |
| V-46 | D | H | D | D | D | H |
| V-47 | D | H | D | D | H | D |
| V-48 | D | H | D | H | D | D |
| V-49 | D | H | D | D | D | D |
| V-50 | D | D | D | D | H | H |
| V-51 | D | D | D | H | D | H |
| V-52 | D | D | D | H | H | D |
| V-53 | D | D | D | D | D | H |
| V-54 | D | D | D | D | H | D |
| V-55 | D | D | D | H | D | D |
| V-56 | D | D | D | D | D | D |

30. The compound according to claim 2, wherein R¹ is deuterium, each of R^{ya}, R^{ya′}, R^{yb} and R^{yb′} is hydrogen, and the compound is selected from any of the compounds in the following table:

| Compound # | R⁴/R^{4′} | R⁵/R^{5′} | R⁶/R^{6′}/R^{6″} | R^{xa} | R^{xb}/R^{xb′} | R^{xc} |
|---|---|---|---|---|---|---|
| V-57 | D | H | H | D | H | H |
| V-58 | D | H | H | H | D | H |
| V-59 | D | H | H | H | H | D |
| V-60 | D | H | H | D | D | H |
| V-61 | D | H | H | D | H | D |
| V-62 | D | H | H | H | D | D |
| V-63 | D | H | H | D | D | D |
| V-64 | H | D | H | D | H | H |
| V-65 | H | D | H | H | D | H |
| V-66 | H | D | H | H | H | D |
| V-67 | H | D | H | D | D | H |
| V-68 | H | D | H | D | H | D |
| V-69 | H | D | H | H | D | D |
| V-70 | H | D | H | D | D | D |
| V-71 | H | H | D | D | H | H |
| V-72 | H | H | D | H | D | H |
| V-73 | H | H | D | H | H | D |
| V-74 | H | H | D | D | D | H |
| V-75 | H | H | D | D | H | D |
| V-76 | H | H | D | H | D | D |
| V-77 | H | H | D | D | D | D |
| V-78 | D | D | H | D | H | H |
| V-79 | D | D | H | H | D | H |
| V-80 | D | D | H | H | H | D |
| V-81 | D | D | H | D | D | H |
| V-82 | D | D | H | H | D | D |
| V-83 | D | D | H | D | H | D |
| V-84 | D | D | H | D | D | D |
| V-85 | H | D | D | D | H | H |
| V-86 | H | D | D | H | D | H |
| V-87 | H | D | D | H | H | D |
| V-88 | H | D | D | D | D | H |
| V-89 | H | D | D | D | H | D |
| V-90 | H | D | D | H | D | D |
| V-91 | H | D | D | D | D | D |
| V-92 | D | H | D | D | H | H |
| V-93 | D | H | D | H | D | H |
| V-94 | D | H | D | H | H | D |
| V-95 | D | H | D | D | D | H |
| V-96 | D | H | D | D | H | D |
| V-97 | D | H | D | H | D | D |
| V-98 | D | H | D | D | D | D |
| V-99 | D | D | D | D | H | H |
| V-100 | D | D | D | H | D | H |
| V-101 | D | D | D | H | H | D |
| V-102 | D | D | D | D | D | H |
| V-103 | D | D | D | D | H | D |
| V-104 | D | D | D | H | D | D |
| V-105 | D | D | D | D | D | D |

31. The compound according to claim 2, wherein each of R⁴, R^{4′}, R⁵, R^{5′}, R⁶, R^{6′}, R^{6″}, R^{xa}, R^{xb}, R^{xb′} and R^{xc} is hydrogen, and the compound is selected from any of the compounds in the following table:

| Compound # | R¹ | R^{ya}/R^{ya′} | R^{yb}/R^{yb′} |
|---|---|---|---|
| VI-1 | D | D | H |
| VI-2 | D | H | D |
| VI-3 | D | D | D |
| VI-4 | D | D | H |
| VI-5 | D | H | D |
| VI-6 | D | D | D |

32. The compound according to claim 2, wherein each of R¹, R^{xa}, R^{xb}, R^{xb′} and R^{xc} is hydrogen, and the compound is selected from any of the compounds in the following table:

| Compound # | R⁴/R^{4′} | R⁵/R^{5′} | R⁶/R^{6′}/R^{6″} | R^{ya}/R^{ya′} | R^{yb}/R^{yb′} |
|---|---|---|---|---|---|
| VI-7 | D | H | H | D | H |
| VI-8 | D | H | H | H | D |
| VI-9 | D | H | H | D | D |
| VI-10 | H | D | H | D | H |
| VI-11 | H | D | H | H | D |
| VI-12 | H | D | H | D | D |
| VI-13 | H | H | D | D | H |
| VI-14 | H | H | D | H | D |
| VI-15 | H | H | D | D | D |
| VI-16 | D | D | H | D | H |
| VI-17 | D | D | H | H | D |
| VI-18 | D | D | H | D | D |
| VI-19 | H | D | D | D | H |
| VI-20 | H | D | D | H | D |
| VI-21 | H | D | D | D | D |
| VI-22 | D | H | D | D | H |
| VI-23 | D | H | D | H | D |
| VI-24 | D | H | D | D | D |
| VI-25 | D | D | D | D | H |
| VI-26 | D | D | D | D | D |
| VI-27 | D | D | D | D | D |

33. The compound according to claim 2, wherein R¹ is deuterium, each of R^{xa}, R^{xb}, R^{xb′} and R^{xc} is hydrogen, and the compound is selected from any of the compounds in the following table:

| Compound # | R⁴/R^{4′} | R⁵/R^{5′} | R⁶/R^{6′}/R^{6″} | R^{ya}/R^{ya′} | R^{yb}/R^{yb′} |
|---|---|---|---|---|---|
| VI-28 | D | H | H | D | H |
| VI-29 | D | H | H | H | D |
| VI-30 | D | H | H | D | D |
| VI-31 | H | D | H | D | H |
| VI-32 | H | D | H | H | D |
| VI-33 | H | D | H | D | D |
| VI-34 | H | H | D | D | H |
| VI-35 | H | H | D | H | D |
| VI-36 | H | H | D | D | D |
| VI-37 | D | D | H | D | H |
| VI-38 | D | D | H | H | D |
| VI-39 | D | D | H | D | D |
| VI-40 | H | D | D | D | H |
| VI-41 | H | D | D | H | D |
| VI-42 | H | D | D | D | D |
| VI-43 | D | H | D | D | H |
| VI-44 | D | H | D | H | D |
| VI-45 | D | H | D | D | D |
| VI-46 | D | D | D | D | H |
| VI-47 | D | D | D | H | D |
| VI-48 | D | D | D | D | D |

34. The compound according to claim 2, wherein each of R¹, R⁴, R^{4′}, R⁵, R^{5′}, R⁶, R^{6′} and R^{6′} is hydrogen, and the compound is selected from any of the compounds in the following table:

| Compound # | R^{xa} | R^{xb}/R^{xb′} | R^{xc} | R^{ya}/R^{ya′} | R^{yb}/R^{yb′} |
|---|---|---|---|---|---|
| VII-1 | D | H | H | D | H |
| VII-2 | D | H | H | H | D |
| VII-3 | D | H | H | D | D |
| VII-4 | H | D | H | D | H |
| VII-5 | H | D | H | H | D |
| VII-6 | H | D | H | D | D |
| VII-7 | H | H | D | D | H |
| VII-8 | H | H | D | H | D |
| VII-9 | H | H | D | D | D |
| VII-10 | D | D | H | D | H |
| VII-11 | D | D | H | H | D |
| VII-12 | D | D | H | D | D |
| VII-13 | D | H | D | D | H |
| VII-14 | D | H | D | H | D |
| VII-15 | D | H | D | D | D |
| VII-16 | H | D | D | D | H |
| VII-17 | H | D | D | H | D |
| VII-18 | H | D | D | D | D |
| VII-19 | D | D | D | D | H |
| VII-20 | D | D | D | H | D |
| VII-21 | D | D | D | D | D |

35. The compound according to claim 2, wherein each of R⁴, R^{4′}, R⁵, R^{5′}, R⁶, R^{6′} and R^{6″} is hydrogen, and the compound is selected from any of the compounds in the following table:

| Compound # | R¹ | R^{xa} | R^{xb}/R^{xb′} | R^{xc} | R^{ya}/R^{ya′} | R^{yb}/R^{yb′} |
|---|---|---|---|---|---|---|
| VII-22 | D | D | H | H | D | H |
| VII-23 | D | D | H | H | H | D |
| VII-24 | D | D | H | H | D | D |
| VII-25 | D | H | D | H | D | H |
| VII-26 | D | H | D | H | H | D |
| VII-27 | D | H | D | H | D | D |
| VII-28 | D | H | H | D | D | H |
| VII-29 | D | H | H | D | H | D |
| VII-30 | D | H | H | D | D | D |
| VII-31 | D | D | D | H | D | H |
| VII-32 | D | D | D | H | H | D |
| VII-33 | D | D | D | H | D | D |
| VII-34 | D | D | H | D | D | H |
| VII-35 | D | D | H | D | H | D |
| VII-36 | D | D | H | D | D | D |
| VII-37 | D | H | D | D | D | H |
| VII-38 | D | H | D | D | H | D |
| VII-39 | D | H | D | D | D | D |
| VII-40 | D | D | D | D | D | H |
| VII-41 | D | D | D | D | H | D |
| VII-42 | D | D | D | D | D | D |

36. The compound according to claim 2, wherein R⁴ and R^{4′} are deuterium, each of R⁵, R^{5′}, R⁶, R^{6′} and R^{6″} is hydrogen, and the compound is selected from any of the compounds in the following table:

| Compound # | R¹ | R^{xa} | R^{xb}/R^{xb′} | R^{xc} | R^{ya}/R^{ya′} | R^{yb}/R^{yb′} |
|---|---|---|---|---|---|---|
| VII-43 | H | D | H | H | D | H |
| VII-44 | H | D | H | H | H | D |
| VII-45 | H | D | H | H | D | D |
| VII-46 | H | H | D | H | D | H |
| VII-47 | H | H | D | H | H | D |
| VII-48 | H | H | D | H | D | D |
| VII-49 | H | H | H | D | D | H |
| VII-50 | H | H | H | D | H | D |
| VII-51 | H | H | H | D | D | D |
| VII-52 | H | D | D | H | D | H |
| VII-53 | H | D | D | H | H | D |
| VII-54 | H | D | D | H | D | D |
| VII-55 | H | D | H | D | D | H |
| VII-56 | H | D | H | D | H | D |
| VII-57 | H | D | H | D | D | D |
| VII-58 | H | H | D | D | D | H |
| VII-59 | H | H | D | D | H | D |
| VII-60 | H | H | D | D | D | D |
| VII-61 | H | D | D | D | D | H |
| VII-62 | H | D | D | D | H | D |
| VII-63 | H | D | D | D | D | D |
| VII-64 | D | D | H | H | D | H |
| VII-65 | D | D | H | H | H | D |
| VII-66 | D | D | H | H | D | D |
| VII-67 | D | H | D | H | D | H |
| VII-68 | D | H | D | H | H | D |
| VII-69 | D | H | D | H | D | D |
| VII-70 | D | H | H | D | D | H |
| VII-71 | D | H | H | D | H | D |
| VII-72 | D | H | H | D | D | D |
| VII-73 | D | D | D | H | D | H |
| VII-74 | D | D | D | H | H | D |
| VII-75 | D | D | D | H | D | D |
| VII-76 | D | D | H | D | D | H |
| VII-77 | D | D | H | D | H | D |
| VII-78 | D | D | H | D | D | D |
| VII-79 | D | H | D | D | D | H |
| VII-80 | D | H | D | D | H | D |
| VII-81 | D | H | D | D | D | D |
| VII-82 | D | D | D | D | D | H |
| VII-83 | D | D | D | D | H | D |
| VII-84 | D | D | D | D | D | D |

37. The compound according to claim 2, wherein R⁵ and R^{5′} are deuterium, each of R⁴, R^{4′} R⁶, R^{6′} and R^{6″} is hydrogen, and the compound is selected from any of the compounds in the following table:

| Compound # | R¹ | R^{xa} | R^{xb}/R^{xb′} | R^{xc} | R^{ya}/R^{ya′} | R^{yb}/R^{yb′} |
|---|---|---|---|---|---|---|
| VII-85 | H | D | H | H | D | H |
| VII-86 | H | D | H | H | H | D |
| VII-87 | H | D | H | H | D | D |
| VII-88 | H | H | D | H | D | H |
| VII-89 | H | H | D | H | H | D |
| VII-90 | H | H | D | H | D | D |
| VII-91 | H | H | H | D | D | H |
| VII-92 | H | H | H | D | H | D |
| VII-93 | H | H | H | D | D | D |
| VII-94 | H | D | D | H | D | H |
| VII-95 | H | D | D | H | H | D |
| VII-96 | H | D | D | H | D | D |
| VII-97 | H | D | H | D | D | H |
| VII-98 | H | D | H | D | H | D |
| VII-99 | H | D | H | D | D | D |
| VII-100 | H | H | D | D | D | H |
| VII-101 | H | H | D | D | H | D |
| VII-102 | H | H | D | D | D | D |
| VII-103 | H | D | D | D | D | H |
| VII-104 | H | D | D | D | H | D |
| VII-105 | H | D | D | D | D | D |
| VII-106 | D | D | H | H | D | H |
| VII-107 | D | D | H | H | H | D |
| VII-108 | D | D | H | H | D | D |
| VII-109 | D | H | D | H | D | H |
| VII-110 | D | H | D | H | H | D |
| VII-111 | D | H | D | H | D | D |
| VII-112 | D | H | H | D | D | H |
| VII-113 | D | H | H | D | H | D |
| VII-114 | D | H | H | D | D | D |
| VII-115 | D | D | D | H | D | H |
| VII-116 | D | D | D | H | H | D |
| VII-117 | D | D | D | H | D | D |
| VII-118 | D | D | H | D | D | H |
| VII-119 | D | D | H | D | H | D |
| VII-120 | D | D | H | D | D | D |
| VII-121 | D | H | D | D | D | H |
| VII-122 | D | H | D | D | H | D |
| VII-123 | D | H | D | D | D | D |
| VII-124 | D | D | D | D | D | H |
| VII-125 | D | D | D | D | H | D |
| VII-126 | D | D | D | D | D | D |

38. The compound according to claim 2, wherein each of R⁶, R^{6′} and R^{6″} is deuterium, each of R⁴, R^{4′}, R⁵ and R^{5′} is hydrogen, and the compound is selected from any of the compounds in the following table:

| Compound # | R¹ | R^{xa} | R^{xb}/R^{xb′} | R^{xc} | R^{ya}/R^{ya′} | R^{yb}/R^{yb′} |
|---|---|---|---|---|---|---|
| VII-127 | H | D | H | H | D | H |
| VII-128 | H | D | H | H | H | D |
| VII-129 | H | D | H | H | D | D |
| VII-130 | H | H | D | H | D | H |
| VII-131 | H | H | D | H | H | D |
| VII-132 | H | H | D | H | D | D |
| VII-133 | H | H | H | D | D | H |
| VII-134 | H | H | H | D | H | D |
| VII-135 | H | H | H | D | D | D |
| VII-136 | H | D | D | H | D | H |
| VII-137 | H | D | D | H | H | D |
| VII-138 | H | D | D | H | D | D |
| VII-139 | H | D | H | D | D | H |
| VII-140 | H | D | H | D | H | D |
| VII-141 | H | D | H | D | D | D |
| VII-142 | H | H | D | D | D | H |
| VII-143 | H | H | D | D | H | D |
| VII-144 | H | H | D | D | D | D |
| VII-145 | H | D | D | D | D | H |
| VII-146 | H | D | D | D | H | D |
| VII-147 | H | D | D | D | D | D |
| VII-148 | D | D | H | H | D | H |
| VII-149 | D | D | H | H | H | D |
| VII-150 | D | D | H | H | D | D |
| VII-151 | D | H | D | H | D | H |
| VII-152 | D | H | D | H | H | D |
| VII-153 | D | H | D | H | D | D |
| VII-154 | D | H | H | D | D | H |
| VII-155 | D | H | H | D | H | D |
| VII-156 | D | H | H | D | D | D |
| VII-157 | D | D | D | H | D | H |
| VII-158 | D | D | D | H | H | D |
| VII-159 | D | D | D | H | D | D |
| VII-160 | D | D | H | D | D | H |
| VII-161 | D | D | H | D | H | D |
| VII-162 | D | D | H | D | D | D |
| VII-163 | D | H | D | D | D | H |
| VII-164 | D | H | D | D | H | D |
| VII-165 | D | H | D | D | D | D |
| VII-166 | D | D | D | D | D | H |
| VII-167 | D | D | D | D | H | D |
| VII-168 | D | D | D | D | D | D |

39. The compound according to claim 2, wherein each of R⁴, R^{4′}, R⁵ and R^{5′} is deuterium, each of R⁶, R^{6′} and R^{6″} is hydrogen, and the compound is selected from any of the compounds in the following table:

| Compound # | R¹ | R^{xa} | R^{xb}/R^{xb′} | R^{xc} | R^{ya}/R^{ya′} | R^{yb}/R^{yb′} |
|---|---|---|---|---|---|---|
| VII-169 | H | D | H | H | D | H |
| VII-170 | H | D | H | H | H | D |
| VII-171 | H | D | H | H | D | D |
| VII-172 | H | H | D | H | D | H |
| VII-173 | H | H | D | H | H | D |
| VII-174 | H | H | D | H | D | D |
| VII-175 | H | H | H | D | D | H |
| VII-176 | H | H | H | D | H | D |
| VII-177 | H | H | H | D | D | D |
| VII-178 | H | D | D | H | D | H |
| VII-179 | H | D | D | H | H | D |
| VII-180 | H | D | D | H | D | D |
| VII-181 | H | D | H | D | D | H |
| VII-182 | H | D | H | D | H | D |
| VII-183 | H | D | H | D | D | D |
| VII-184 | H | H | D | D | D | H |
| VII-185 | H | H | D | D | H | D |
| VII-186 | H | H | D | D | D | D |
| VII-187 | H | D | D | D | D | H |
| VII-188 | H | D | D | D | H | D |
| VII-189 | H | D | D | D | D | D |
| VII-190 | D | D | H | H | D | H |
| VII-191 | D | D | H | H | H | D |
| VII-192 | D | D | H | H | D | D |
| VII-193 | D | H | D | H | D | H |
| VII-194 | D | H | D | H | H | D |
| VII-195 | D | H | D | H | D | D |
| VII-196 | D | H | H | D | D | H |
| VII-197 | D | H | H | D | H | D |
| VII-198 | D | H | H | D | D | D |
| VII-199 | D | D | D | H | D | H |
| VII-200 | D | D | D | H | H | D |
| VII-201 | D | D | D | H | D | D |
| VII-202 | D | D | H | D | D | H |
| VII-203 | D | D | H | D | H | D |
| VII-204 | D | D | H | D | D | D |
| VII-205 | D | H | D | D | D | H |
| VII-206 | D | H | D | D | H | D |
| VII-207 | D | H | D | D | D | D |
| VII-208 | D | D | D | D | D | H |
| VII-209 | D | D | D | D | H | D |
| VII-210 | D | D | D | D | D | D |

40. The compound according to claim 2, wherein each of R⁴, R^{4′}, R⁶, R^{6′}, and R^{6″} is deuterium, each of R⁵ and R^{5′} is hydrogen, and the compound is selected from any of the compounds in the following table:

| Compound # | R¹ | R^{xa} | R^{xb}/R^{xb′} | R^{xc} | R^{ya}/R^{ya′} | R^{yb}/R^{yb′} |
|---|---|---|---|---|---|---|
| VII-211 | H | D | H | H | D | H |
| VII-212 | H | D | H | H | H | D |
| VII-213 | H | D | H | H | D | D |
| VII-214 | H | H | D | H | D | H |
| VII-215 | H | H | D | H | H | D |
| VII-216 | H | H | D | H | D | D |
| VII-217 | H | H | H | D | D | H |
| VII-218 | H | H | H | D | H | D |
| VII-219 | H | H | H | D | D | D |
| VII-220 | H | D | D | H | D | H |
| VII-221 | H | D | D | H | H | D |
| VII-222 | H | D | D | H | D | D |
| VII-223 | H | D | H | D | D | H |
| VII-224 | H | D | H | D | H | D |
| VII-225 | H | D | H | D | D | D |
| VII-226 | H | H | D | D | D | H |
| VII-227 | H | H | D | D | H | D |
| VII-228 | H | H | D | D | D | D |
| VII-229 | H | D | D | D | D | H |
| VII-230 | H | D | D | D | H | D |
| VII-231 | H | D | D | D | D | D |
| VII-232 | D | D | H | H | D | H |
| VII-233 | D | D | H | H | H | D |
| VII-234 | D | D | H | H | D | D |
| VII-235 | D | H | D | H | D | H |
| VII-236 | D | H | D | H | H | D |
| VII-237 | D | H | D | H | D | D |
| VII-238 | D | H | H | D | D | H |
| VII-239 | D | H | H | D | H | D |
| VII-240 | D | H | H | D | D | D |
| VII-241 | D | D | D | H | D | H |
| VII-242 | D | D | D | H | H | D |
| VII-243 | D | D | D | H | D | D |
| VII-244 | D | D | H | D | D | H |
| VII-245 | D | D | H | D | H | D |
| VII-246 | D | D | H | D | D | D |
| VII-247 | D | H | D | D | D | H |
| VII-248 | D | H | D | D | H | D |
| VII-249 | D | H | D | D | D | D |
| VII-250 | D | D | D | D | D | H |
| VII-251 | D | D | D | D | H | D |
| VII-252 | D | D | D | D | D | D |

41. The compound according to claim 2, wherein each of R⁵, R^{5′}, R⁶, R^{6′} and R^{6″} is deuterium, each of R⁴ and R^{4′} is hydrogen, and the compound is selected from any of the compounds in the following table:

| Compound # | R¹ | R^{xa} | R^{xb}/R^{xb′} | R^{xc} | R^{ya}/R^{ya′} | R^{yb}/R^{yb′} |
|---|---|---|---|---|---|---|
| VII-253 | H | D | H | H | D | H |
| VII-254 | H | D | H | H | H | D |
| VII-255 | H | D | H | H | D | D |
| VII-256 | H | H | D | H | D | H |
| VII-257 | H | H | D | H | H | D |
| VII-258 | H | H | D | H | D | D |
| VII-259 | H | H | H | D | D | H |
| VII-260 | H | H | H | D | H | D |
| VII-261 | H | H | H | D | D | D |
| VII-262 | H | D | D | H | D | H |
| VII-263 | H | D | D | H | H | D |
| VII-264 | H | D | D | H | D | D |
| VII-265 | H | D | H | D | D | H |
| VII-266 | H | D | H | D | H | D |
| VII-267 | H | D | H | D | D | D |
| VII-268 | H | H | D | D | D | H |
| VII-269 | H | H | D | D | H | D |
| VII-270 | H | H | D | D | D | D |
| VII-271 | H | D | D | D | D | H |
| VII-272 | H | D | D | D | H | D |
| VII-273 | H | D | D | D | D | D |
| VII-274 | D | D | H | H | D | H |
| VII-275 | D | D | H | H | H | D |
| VII-276 | D | D | H | H | D | D |
| VII-277 | D | H | D | H | D | H |
| VII-278 | D | H | D | H | H | D |
| VII-279 | D | H | D | H | D | D |
| VII-280 | D | H | H | D | D | H |
| VII-281 | D | H | H | D | H | D |
| VII-282 | D | H | H | D | D | D |
| VII-283 | D | D | D | H | D | H |
| VII-284 | D | D | D | H | H | D |
| VII-285 | D | D | D | H | D | D |
| VII-286 | D | D | H | D | D | H |
| VII-287 | D | D | H | D | H | D |
| VII-288 | D | D | H | D | D | D |
| VII-289 | D | H | D | D | D | H |
| VII-290 | D | H | D | D | H | D |
| VII-291 | D | H | D | D | D | D |
| VII-292 | D | D | D | D | D | H |
| VII-293 | D | D | D | D | H | D |
| VII-294 | D | D | D | D | D | D |

42. The compound according to claim 2, wherein each of R⁴, R^{4′}, R⁵, R^{5′}, R⁶, R^{6′} and R^{6″} is deuterium, and the compound is selected from any of the compounds in the following table:

| Compound # | R¹ | R^{xa} | R^{xb}/R^{xb′} | R^{xc} | R^{ya}/R^{ya′} | R^{yb}/R^{yb′} |
|---|---|---|---|---|---|---|
| VII-295 | H | D | H | H | D | H |
| VII-296 | H | D | H | H | H | D |
| VII-297 | H | D | H | H | D | D |
| VII-298 | H | H | D | H | D | H |
| VII-299 | H | H | D | H | H | D |
| VII-300 | H | H | D | H | D | D |
| VII-301 | H | H | H | D | D | H |
| VII-302 | H | H | H | D | H | D |
| VII-303 | H | H | H | D | D | D |
| VII-304 | H | D | D | H | D | H |
| VII-305 | H | D | D | H | H | D |
| VII-306 | H | D | D | H | D | D |
| VII-307 | H | D | H | D | D | H |
| VII-308 | H | D | H | D | H | D |
| VII-309 | H | D | H | D | D | D |
| VII-310 | H | H | D | D | D | H |
| VII-311 | H | H | D | D | H | D |
| VII-312 | H | H | D | D | D | D |
| VII-313 | H | D | D | D | D | H |
| VII-314 | H | D | D | D | H | D |
| VII-315 | H | D | D | D | D | D |
| VII-316 | D | D | H | H | D | H |
| VII-317 | D | D | H | H | H | D |
| VII-318 | D | D | H | H | D | D |
| VII-319 | D | H | D | H | D | H |
| VII-320 | D | H | D | H | H | D |
| VII-321 | D | H | D | H | D | D |
| VII-322 | D | H | H | D | D | H |
| VII-323 | D | H | H | D | H | D |
| VII-324 | D | H | H | D | D | D |
| VII-325 | D | D | D | H | D | H |
| VII-326 | D | D | D | H | H | D |
| VII-327 | D | D | D | H | D | D |
| VII-328 | D | D | H | D | D | H |
| VII-329 | D | D | H | D | H | D |
| VII-330 | D | D | H | D | D | D |
| VII-331 | D | H | D | D | D | H |
| VII-332 | D | H | D | D | H | D |
| VII-333 | D | H | D | D | D | D |
| VII-334 | D | D | D | D | D | H |
| VII-335 | D | D | D | D | H | D |
| VII-336 | D | D | D | D | D | D |

43. A composition comprising a compound according to any one of claims 1-42 and a pharmaceutically acceptable adjuvant, carrier, or vehicle.

44. The composition according to claim 43, in combination with an additional therapeutic agent.

45. The composition according to claim 44, wherein the additional therapeutic agent is a chemotherapeutic agent.

46. A method for inhibiting BTK, or a mutant thereof, activity in a patient or in a biological sample comprising the step of administering to said patient or contacting said biological sample with a compound according to any one of claims 1-42.

47. The method according to claim 46, wherein the BTK, or a mutant thereof, activity is inhibited irreversibly.

48. The method according to claim 47, wherein the BTK, or a mutant thereof, activity is inhibited irreversibly by covalently modifying Cys 481 of BTK.

49. A method for treating a BTK-mediated disorder in a patient in need thereof, comprising the step of administering to said patient a compound according to any one of claims 1-42 or a composition according to claim 43.

50. The method according to claim 49, wherein the disorder is an autoimmune disease, a heteroimmune disease, an inflammatory disease, a cancer, a disease of the bone and joints, or a thromboembolic disorder.

51. The method according to claim 50, wherein the disorder is selected from rheumatoid arthritis, multiple sclerosis, B-cell chronic lymphocytic leukemia, acute lymphocytic leukemia, hairy cell leukemia, non-Hodgkin's lymphoma, Hodgkin's lymphoma, multiple myeloma, bone cancer, bone metastasis, osteoporosis, irritable bowel syndrome, Crohn's disease, lupus, or disorders associated with renal transplant.
